(19) United States
(12) Patent Application Publication
(10) Pub. No.: US 2011/0200552 A1
(43) Pub. Date: Aug. 18, 2011

(54) CYTOKINE DESIGN

(75) Inventors: Carlos Ricardo Rodrigues Dos Reis, Gronigen (NL); Wilhelmus Johannes Quax, Kropswolde (NL); Afshin Samali, Galway (IE); Eva Szegezdi, Galway (IE); Robbert Hans Cool, Groningen (NL); Albert Martinus Van Der Sloot, Barcelona (ES); Luis Serrano, Barcelona (ES); Vicente Tur, Barcelona (ES)

(73) Assignees: RIJKSUNIVERSITEIT GRONINGEN, Groningen (NL); NATIONAL UNIVERSITY OF IRELAND, GALWAY, Galway (IE); TRISKEL THERAPEUTICS B.V, Gronigen (NL); Fundacio Privada Centre De Regulacio Genomica (CRG), Barcelona, ES (unknown); Institucio Catalana De Recerca I Estudis Avancats (ICREA), Barcelona (ES)

(21) Appl. No.: 12/744,069

(22) PCT Filed: Nov. 21, 2008
(86) PCT No.: PCT/IB08/03476

§ 371 Date: Dec. 22, 2010

(30) Foreign Application Priority Data
Nov. 23, 2007 (GB) 0723059.2

(51) Int. Cl. A61K 38/19; C07K 14/525; C07H 21/04; A61P 35/00; C12N 5/10; C12N 1/00; C12N 5/071

(52) U.S. Cl. 424/85.1; 530/351; 536/23.5; 435/325; 435/419; 435/243; 435/375

(57) ABSTRACT

The present invention relates to novel cytokines, which have a modified selectivity/specificity for their cognate receptors. In particular, the invention relates to a variant TRAIL protein, which has superior selectivity for the death receptor 4 (TRAIL-RI) over the death receptor 5 (TRAIL-R2). In addition, the invention relates to a variant TRAIL protein which exhibits superior selectivity for the death receptor 4 (TRAIL-R1) over the decoy receptors DcR1 (TRAIL-R3) and DcR2 (TRAIL-R4).

The present invention relates to novel cytokines, which have a modified selectivity/specificity for their cognate receptors.

Cytokines are a family of growth factors, secreted primarily from leukocytes, and are messenger proteins that act as potent regulators capable of effecting cellular processes at subnanomolar concentrations. Their size allows cytokines to be quickly transported around the body and degraded when required. Their role in controlling a wide range of cellular functions, especially the immune response and cell growth, has been revealed by extensive research over the last twenty years¹. These roles include immune response regulation², inflammation³, wound healing⁴, embryogenesis and development, and apoptosis⁵.

Clinical use of cytokines to date has focused on their role as regulators of the immune system ⁶, for instance in promoting a response against thyroid cancer⁷. Their control of cell growth and differentiation has also made cytokines anti-cancer targets^{8,9}. Novel mutations in cytokines and cytokine receptors have been shown to confer disease resistance in some cases¹⁰. The creation of synthetic cytokines (muteins) in order to modulate activity and remove potential side effects has also been an important avenue of research¹¹.

Cytokine molecules have thus been shown to play a role in diverse physiological functions, many of which play a role in disease processes. Alteration of their activity or specificity is a means to alter the disease phenotype and as such, the identification of novel cytokine molecules is of significant scientific interest.

Of particular interest are ligands that belong to the Tumor Necrosis Factor ligand (TNF) family. These proteins are involved in a wide range of biological activities, ranging from cell proliferation to apoptosis.

TRAIL, a member of the TNF ligand family, in its soluble form, selectively induces apoptosis in tumour cells in vitro and in vivo. Unlike other apoptosis inducing TNF family members, TRAIL appears to be inactive against normal healthy tissue, therefore attracting great interest as a potential cancer therapeutic¹². Therefore TRAIL has the potential to serve as a safe and potent therapeutic agent against tumour cells. A number of in vitro studies have shown that many tumour cell lines of divergent origins including cancers of the lung, breast, prostate, bladder, kidney, ovarian and colon as well as melanoma, leukemia and multiple myeloma, are sensitive to TRAIL induced apoptosis.

It has also been reported that TRAIL may play an important role in immune system modulation, including autoimmune diseases such as rheumatoid arthritis.

A recent significant publication has shown that TRAIL-R3 (DcR1) is upregulated by p53 in breast tumour cells through use of the genotoxic drug, doxorubicin¹³. This implies that efficacy of wild-type (WT) TRAIL may be diminished in anti-tumour therapy since it also binds the decoy receptors (that do not initiate apoptosis). Therefore, variants of TRAIL that have altered selectivity/specificity could bind directly to the pro-apoptotic receptors DR4 (death receptor 4, TRAIL-R1) or DR5 (death receptor 5, TRAIL-R2). Furthermore such variants could escape binding by the decoy receptors, and remain available for the apoptotosis-inducing receptors DR4 or DR5 and might, in theory, have ultimately improved application in cancer treatment.

In this respect, selectivity of novel molecules is of primary importance to discern the specific role of the activation of different receptors and therefore the functional effects of ligand binding to several receptors, and the concomitant influence on the pathogenesis of the associated diseases related to signal activation.

### SUMMARY OF THE INVENTION

According to a first aspect of the invention, there is provided a variant TRAIL protein, which has superior selectivity for the death receptor 4 (TRAIL-R1) over the death receptor 5 (TRAIL-R2).

TRAIL (also known as TNFSF10, TL2; APO2L; CD253; Apo-2L) is a member of the TNF ligand family^{14 15} and an example of a cytokine that binds to more than one receptor, including decoy receptors which lack or have truncated intracellular domains. TRAIL is a promiscuous ligand as it binds to five cognate receptors of the TNF receptor family; to the death receptor 4 (DR4, TRAIL-R1), death receptor 5 (DR5, TRAIL-R2, KILLLER, TRICK-2) and to the decoy receptor 1, (DcR1, TRAIL-R3, TRIDD), decoy receptor 2 (DcR2, TRAIL-R4, TRUNDD) and to the soluble secreted receptor osteoprotegerin (OPG). Only DR4 (TRAIL-R1) and DR5 (TRAIL-R2), and not DcR1, DcR2 or OPG contain a functional death domain (DD) and thus, only binding of TRAIL to these receptors induces apoptosis via activation of a cell—extrinsic or death receptor-mediated apoptosis pathway. Through the same receptors as well as through DcR2, TRAIL also appears to be able to induce the pro-survival NF-κB pathway. The factors that determine which pathway (apoptosis or survival) dominates in a given cell are poorly understood.

It is known that receptors DR4 and/or DR5 may be up-regulated after treatment with DNA damaging chemotherapeutic drugs. In such cells, chemotherapeutics can significantly increase the response to TRAIL-induced apoptosis. It has been suggested in the literature that DR5 is the principal receptor that transmits the death signal. However, the inventors are of the view that in at least some cancer cells the apoptotic signal is primarily transmitted by DR4. For this reason, such cancers are therefore less responsive to WT TRAIL, as it has a higher affinity for DR5. Examples of such cancers include, but are not limited to, chronic lymphocytic leukaemia and mantle cell lymphoma²⁷.

For this reason, having selective inducers of DR4 (TRAIL-R1) signalling is of great interest. The inventors are therefore of the view that the use of DR4-receptor selective TRAIL variants could permit better therapies with higher efficacy and possibly less side effects as compared to wild-type TRAIL.

Additionally to its superior selectivity for the death receptor 4 over death receptor 5, a TRAIL variant according to the invention preferably exhibits superior selectivity for the death receptor 4 (TRAIL-R1) over the decoy receptors DcR1 (TRAIL-R3) and DcR2 (TRAIL-R4) and OPG. DcR1 and OPG do not contain a death domain and DcR2 contain a truncated death domain. Binding of TRAIL to these receptors does not induce apoptosis; on the contrary, it may actually prevent apoptosis by sequestering available TRAIL from DR4 and DR5, or by leading to NF-κB activation via DcR2. For this reason, it is preferred that the TRAIL variants of the invention are not sequestered via this route.

The variant TRAIL molecules of the present invention are of great utility in inducing apoptosis in cells. Apoptosis may be induced in vivo, ex vivo or in vitro. Preferably, apoptosis is induced in cancerous cells, and not in healthy cells.

By “variant” TRAIL protein is meant that the TRAIL protein differs in at least one amino acid position, (including two, three, four, five, six or more one amino acid positions) from the WT TRAIL protein (also known as TNFSF10, TL2; APO2L; CD253; Apo-2L), Entrez GeneID: 8743; accession number NM_{—}003810.2; UniProtKB/Swiss-Prot: P50591; UniProtKB/TrEMBL: Q6IBA9.

By“selectivity” is meant that the variants of the invention have substantially greater affinity for DR4 than their affinity for DR5 in relative terms, and preferably also for the decoy receptors DcR1 (TRAIL-R3) and DcR2 (TRAIL-R4). By “substantially greater affinity” we mean that there is a measurably higher affinity of the TRAIL variant for DR4 as compared with its affinity for DR5, DcR1, DcR2 and OPG. Preferably, the affinity is at least 1.5-fold, 2-fold, 5-fold, 10-fold, 100-fold, or even 1000-fold or greater for DR4 than for one or more of DR5, DcR1, DcR2 and OPG. More preferably, the affinity is at least 1.5-fold, 2-fold, 5-fold, 10-fold, 100-fold, or even 1000-fold or greater for DR4 than for all of DR5, DcR1, DcR2 and OPG. Methods for measuring the binding affinity of proteins for binding partners are well known in the art, including for example, competition assays, Surface Plasmon Resonance and so on.

Preferably, the binding affinity of the TRAIL variants of the invention for DR4 is also higher in absolute terms than the binding affinity of the WT TRAIL molecule for DR4. Furthermore, the binding affinity of the TRAIL variants of the invention for DR5 should be lower than the binding affinity of the WT TRAIL molecule for DR5. The same is preferably true of the binding affinity demonstrated for DcR1, DcR2 and OPG.

Preferably, a TRAIL variant according to the invention displays a binding affinity of less than Kd=1000 nM for DR4, preferably less than Kd=100 nM, more preferably less than Kd=10 nM, more preferably less than Kd=5 nM, more preferably less than Kd=1 nM, measured by surface plasmon resonance as described before (Van der Sloot, et. al.¹⁶) where the affinity of WT TRAIL was measured to be 7.2 nM.

Preferably, a TRAIL variant according to the invention displays a binding affinity of more than Kd=1 nM for DR5, preferably more than Kd=5 nM, preferably more than Kd=10 nM, preferably more than Kd=100 nM, more preferably more than Kd=1000 nM measured by surface plasmon resonance as described before (Van der Sloot, et. al.¹⁶) where the affinity of WT TRAIL was measured to be 2.5 nM.

Preferably, a TRAIL variant according to the invention displays a binding affinity of more than Kd=10 nM for DcR1, preferably more than Kd=25 nM, preferably more than Kd=50 nM, preferably more than Kd=100 nM, more preferably more than Kd=1000 nM measured by surface plasmon resonance as described before (Van der Sloot, et. al.¹⁶) where the affinity of WT TRAIL was measured to be about 25 nM.

Preferably, a TRAIL variant according to the invention displays a binding affinity of more than Kd=10 nM for DcR2, preferably more than Kd=25 nM, preferably more than Kd=50 nM, preferably more than Kd=100 nM, more preferably more than Kd=1000 nM measured by surface plasmon resonance as described before (Van der Sloot, et, al.¹⁶) where the affinity of WT TRAIL was measured to be about 8 nM.

Particular examples of suitable residues for mutation include, but are not limited to, the following positions: 131, 199, 201, 204, 215 and 218. Further examples of suitable residues are shown in table 3, namely, at positions 127, 129, 131, 133, 143, 144, 149, 155, 157, 159, 160, 161, 162, 179, 193, 194, 197, 198, 199, 201, 202, 203, 204, 205, 207, 212, 214, 215, 216, 218, 221, 224, 232, 239, 249, 251, 252, 261, 262, 263, 264, 265, 269, 270, 271, 272. Of these mutations at residues 149, 159, 193, 201, 212 and 251 are preferred.

All references to amino acid positions in the TRAIL protein sequence presented herein, and to specific TRAIL mutants are intended to refer to the amino acid sequence given in SEQ ID NO:1. One aspect of the invention relates to a cytokine that is mutated at one, two, three, four, five, six, seven, eight, nine, ten, eleven or more of these positions. These residues are preferred because they represent amino acids whose side-chains are predicted by the inventors to fit into the binding interface of DR4, at points that are not conserved between the different receptors or at points unique to DR4.

The inventors have identified a number of preferred mutants, which are shown in table 3. These mutants were all chosen because they have increased affinity for the DR4 receptor and/or decreased binding to the DR5 receptor. Mutants which were not predicted to be stable were not included in the table. A description of how these mutants were designed, how their binding affinity was determined and how the stability of these mutants was assessed can be found in example 2. Preferred are mutations at positions 149, 159, 193, 201, 204, 212, 215 and 251. Particularly preferred are the following mutations R149I, R149M, R149N, R149K, S159R, Q193H, Q193K, N199R, K201R, K204D, K204E, K204L, K204Y, K212R, S215D, S215E, S215H, S215K, K251D, K251E and K251Q.

In one embodiment of the invention, TRAIL variants comprising combinations of the mutants in table 3 are provided. Such combinations can include 2, 3, 4, 5, 6 or more of the mutations of table 3. In a further embodiment of the invention, the TRAIL variant can contain combinations of mutations that comprise the mutations of table 3 and further mutations disclosed herein.

As the skilled person will understand, the amino acid changes have to be sufficient to alter the binding specificity of WT TRAIL. It is therefore preferred that an amino acid is substituted for another amino acid with different physiochemical properties. Such substitutions include, but are not limited to, substituting basic amino acids with acidic amino acids or substituting acidic amino acids with basic amino acids. It is further preferred that the amino acid substitution is non-conservative. Such a substitution is more likely to result in an altered binding specificity of the protein since the charge and/or the structure in the area surrounding the mutation is likely to be altered. However, the use of protein design algorithms, like FoldX, also allows one to evaluate the effects of substituting amino acids with amino acids having comparable physiochemical properties (e.g. K201R). For example, substituting a particular amino acid with an amino acid with similar physiochemical properties but having a different size can optimise the interaction with one receptor while interactions with the other receptor become less optimal.

A grouping of amino acids based on physiochemical properties is given in the table below. Amino acids with comparable physiochemical properties are shown in the same row with the one letter abbreviation of amino acids given in brackets. Amino acid substitutions in the same row are conservative:

TABLE 1

| | |
|---|---|
| Amino acids with aliphatic | Glycine (G), alanine (A), valine (V), |
| side-chains (R groups) | leucine (L), isoleucine (I), proline (P) |
| Non-aromatic amino acids | Serine (S), threonine (T) |
| with hydroxyl r groups | |
| Amino acids with polar | Asparagine (N), glutamine (Q) |
| R-groups | |
| Amino acids with | Cysteine (C), methionine (M) |
| sulphur-containing R-groups | |
| Amino acids with acidic | Aspartate (D), glutamate (E) |
| R-groups | |
| Amino acids with basic | Arginine (R), Lysine (K), histidine (H) |
| R-groups | |
| Amino acids with aromatic ring | Phenylalanine (F), tyrosine (Y), |
| R-groups | tryptophan (W) |

Equivalent substitutions at the above positions, with a specific type of residue are preferred. For example, preferred mutations at positions 131, 199 and 201 are to basic amino acids. Particularly preferred at 131 are mutations to arginine or lysine. The mutant G131R, for example, shows superior binding to DR4, compared to WT TRAIL, over a large range of physiological concentrations of the ligand. In comparison, the increase in affinity to DR5 is less evident (FIG. 5).

Based on the FoldX analysis the following mutation sites in WT TRAIL increasing DR4-selectivity are predicted by the inventors:

TABLE 2

| Site in WT TRAIL | Preferred mutation to: |
|---|---|
| Glycine at position 131 | Arginine or lysine |
| Arginine at position 149 | Isoleucine or methionine or asparagine |
| | or lysine |
| Serine at position 159 | arginine |
| Glutamine at position 193 | histidine or lysine |
| Asparagine at position 199 | arginine or histidine |
| Lysine at position 201 | histidine or arginine |
| Lysine at position 204 | acidic amino acids (D, E) or leucine |
| | or tyrosine |
| Lysine at position 212 | arginine |
| Serine at position 215 | acidic or to basic amino acids (D, E, H, K) |
| Aspartic Acid at position 218 | basic or aromatic amino acids (H, Y, K, F) |
| Lysine at position 251 | Acidic amino acids (D, E) or glutamine |

Preferred mutations in position 131 are to basic residues arginine and lysine (FIG. 39). Preferred mutations at position 149 are to isoleucine or methionine or asparagine or lysine. These mutations are predicted to result in a decrease in affinity for DR5 while not affecting the affinity for DR4 (FIG. 4). At position 159, the preferred mutation is to arginine. According to the prediction, this mutation should result in a substantial decrease of the affinity for DR5. Mutations to histidine and lysine are the preferred mutations at position 193, both of which ought to result in a increase in relatively small affinity for DR4, where mutation to histidine is predicted to cause an additional decrease in affinity of DR5 (FIG. 4). Preferred mutations at position 199 are to arginine or histidine. The mutation to arginine, for example, is predicted to result in increased affinity for DR4 while the affinity for DR5 is decreased (FIG. 4). Preferred mutations at position 201 are to histidine or arginine. The mutation of K201 to arginine, for example, is predicted to increase affinity for DR4 while the affinity for DR5 is reduced (FIG. 1). Preferred mutations at position 204 are to acidic amino acids, particularly aspartic acid or glutamic acid, or leucine or tyrosine. These mutants were predicted to have a reduced affinity for DR5, with an accompanying small increased affinity for DR4 for the mutations to acidic residues (FIG. 4). Mutation of residue Lys212 to arginine is predicted to result in a small increase in affinity for DR4.

At position 215, mutations may be to acidic or to basic amino acids; glutamic acid, histidine, lysine and aspartic acid are preferred. Mutations of S215 to glutamic acid and lysine were predicted to increase the affinity of the TRAIL variant to the DR4 receptor, while the affinity to DR5 is decreased (FIG. 4). Mutation of the residue to aspartic acid is predicted to result in a dramatic decrease of the DR5 binding affinity of the TRAIL variant, while its affinity for the DR4 receptor is predicted to be only slightly reduced (FIG. 4).

At position 218, mutations may be to basic or aromatic amino acids; histidine, tyrosine, glutamic acid, lysine and phenylalanine are preferred. These mutations are all characterised by an increased affinity for the DR4 receptor, in conjunction with reduced binding ability to DR5. D218H, in particular, shows unaltered binding to DR4, while it's binding affinity to DR5 is reduced 1.5-3 times (FIG. 2A). These mutants also show in comparison with WT TRAIL a higher dissociation rate of the complex formed with DR5 whereas that of the DR4 complex is hardly affected (data not shown). Furthermore, the mutants D218H and D218Y also show reduced binding to the decoy receptors DcR1 and DcR2 and OPG (FIG. 2B, 2C).

Preferred mutations at position 251 are to acidic amino acids (aspartic acid or glutamic acid), or glutamine. These mutants were predicted to have a reduced affinity for DR5, (FIG. 4)

These mutants are all preferred because they show increased affinity and/or selectivity for the DR4 (TRAIL-R1) receptor in computational design models (e.g. FoldX). Some of these mutants further show decreased binding to DR5 (TRAIL-R2). Preferred mutations with such properties, as identified so far include G131R, G131K, R149I, R149M, R149N, R149K, S159R, Q193H, Q193K, N199H, N199R, K201H, K201R, K204D, K204E, K204L, K204Y, K212R, S215E, S215D, S215H, S215K, D218Y, D218H, K251D, K251E and K251Q. Most preferred are the mutants G131R, G131K, R149I, S159R, N199H, N199R, K201H, K201R, K204E, K204D, S215E, S215H, S215K, S215D, D218Y and D218H.

Many of these mutations are predicted to show superior binding to the DR4 receptor and, in case of mutations Q192H, Q193K, N199R, K201R, K204D, K204E, K212R, S215E, S215H and S215K, also reduced binding to the DR5 receptor. The mutation K201R resulted in increased binding of the mutant TRAIL to, both, DR4 and DR5. However, the increase in binding to DR4 is much higher than the increase in binding to DR5. The mutation S215D results in a TRAIL protein whose ability to bind DR5 is greatly reduced, while the binding affinity to DR4 remains almost unaltered.

Combinations of mutations are also envisioned. Preferred combinations are of 2, 3, 4, 5, 6, 7, 8 or all 9 mutations at positions 131, 149, 159, 193, 199, 201, 204, 215 and 218. Preferred double mutations include 199 plus 201 and 131 plus 218. Particularly preferred combinations are N199R/K201H, N199H/K201R and G131R/D218H. Preferred triple mutations include 131, 199 and 201. A preferred example of such a triple combination is G131R/N199R/K201H. This mutant shows a much increased affinity for the DR4 receptor when compared to WT TRAIL or the single mutant G131R, whereas the increased affinity to DR5, compared to WT TRAIL, is much less evident (FIG. 5). Further preferred combinations comprise combinations of the triple combination G131R/N199R/K201H with mutations in one or more positions of the list: 149, 159, 193, 204, 212, 215, 218, and 251. More specifically, the triple combination can be combined with one or more mutations of the following list: R149I, S159R, S215D.

Further preferred combinations comprise combinations of S159R with one or more mutations of the list: 149, 193, 204, 212, 215, 218, and 251. More specifically, S159R can be combined with one or more mutations of the list: R149I, S215D.

These combinations are preferred because of their superior affinity and/or selectivity to DR4 (TRAIL-R1) compared to single mutations. Other combinations which show similar effects are also included in an embodiment of the present invention.

The above mutations may be introduced in the full length TRAIL sequence. Preferably, however, the above mutations are introduced into soluble forms of the TRAIL sequence, such as forms comprising amino acids 114-281 or comprising amino acids 95-281; other examples will be clear to those of skill in the art. Preferred TRAIL variants according to the invention are thus variants of the soluble fragments of the full length TRAIL sequence given in SEQ ID NO:1.

A preferred soluble fragment template comprises amino acids 114-281 (herein termed rhTRAIL) and all mutants described herein are of this length. The WT TRAIL sequence of rhTRAIL (114-281) preceded by a methionine is presented in SEQ ID NO: 3 and a preferred coding sequence is presented in SEQ ID NO:4; variants of the invention may thus be derived from this sequence.

However, as the skilled reader will appreciate, variations in these soluble templates will very likely retain the properties of this soluble form and show biological activity if additional residues C terminal and/or N terminal of these boundaries in the polypeptide sequence are included. For example, an additional 1, 2, 3, 4, 5, 10, 20 or even 30 or more amino acid residues from the WT TRAIL sequence, or from a homologous sequence, may be included at either or both the C terminal and/or N terminal of these boundaries, without prejudicing the ability of the polypeptide fragment to fold correctly and exhibit biological activity. Similarly, truncated variants of this template in which one or a few amino acid residues (for example, 1, 2, 3, 4, 5, 10 or more) may be deleted at either or both the C terminus or the N terminus without prejudicing biological activity.

TRAIL variants according to the invention preferably function to induce apoptosis in target cells. By “induces apoptosis” is meant that a compound according to the present invention acts to cause cell death in target cells. A compound according to the invention may induce apoptosis in vivo and/or in vitro.

Apoptosis can be measured by a number of different assays, as will be clear to those of skill in the art. Examples include DNA laddering assays (see, for example, EP0835305; Immunex); detection of chromatin fragmentation and condensation with Hoechst33342, staining and detection of phosphatidyl serine exposure in combination with membrane permeabilisation measured by staining with Annexin V and propidium iodide. What these assays share in common is a measurement of biochemical or morphological changes occurring in dying cells upon sustained contact with a concentration of the compound whose activity is being measured. Cell death can be expressed as an increase of the percentage of dying cells in response to exposure to the compound (i.e. the percentage of dying cells in untreated, control cell population is subtracted from that of in cell populations exposed to the drug). Effective concentrations are typically calculated in the form of IC50 values, which is the concentration of compound at which 50% of the cells undergo apoptosis. Preferably, a compound according to the invention induces apoptosis in 50% of cells at a concentration of between 1 ng/ml and 1000 ng/ml, more preferably between 1 ng/ml and 100 ng/ml, more preferably between 1 ng/ml and 10 ng/ml.

Preferably, according to the invention, useful compounds possess IC50 values of between 1 ng/ml and 1000 ng/ml, more preferably between 1 ng/ml and 100 ng/ml, more preferably between 1 ng/ml and 10 ng/ml.

Alternatively, apoptosis can also be measured by caspase activation and other assays known to those of skill in the art.

TRAIL variants according to the invention induce apoptosis in a variety of target cells. Target cells are cancerous cells and do not include healthy cells. Cancerous cells targeted by the variants of the invention express the DR4 receptor on their cell surface and this marks them out as targets for the variants of the present invention. Preferred target cells include ovarian cancer cells, breast cancer cells, lung cancer cells, leukemias (for example acute lymphocytic leukemia (ALL), acute myelogenous leukemia (AML), chronic lymphocytic leukemia (CLL) and chronic myelogenous leukemia (CML), lymphomas (for example mantle cell lymphoma), Burkitt's lymphoma (BJAB) and so on. Other examples will be known to those of skill in the art. Target cells may preferably be characterised by the relative level of expression of certain cell surface markers including DR4 and DR5.

The specific effect of the compounds according to the invention on target cells can be evaluated by exposing different types of cell to concentrations of the compound at ranges either side of its IC50 value and assessing the degree of cell death elicited. In reality, because of the fairly precise requirements that cells possess for particular growth conditions, it is true that most compounds will cause a degree of apoptosis at some concentration. Accordingly, when it is said that compounds according to the invention cause apoptosis specifically in target cells it is meant that there is a concentration window within which apoptosis is caused only in these target cells and in this window there is no adverse effect on other types of cell.

Suitable mutations as listed above have been identified using the methodology set out below. Of course, this methodology may be used iteratively to select further interesting mutations to combine with those already listed and set out herein.

The method preferably involves substituting residues in the TRAIL ligand for replacement residues that include amino acid side-chain conformations that are predicted to fit into the binding interface with the target receptor so as to provide an increase in binding affinity for that receptor. The inventors have used the computer design algorithm FoldX, which performs inverse folding. Briefly, this algorithm decorates a fixed backbone structure with amino acid side chains from a rotamer library. FoldX thus performs a rotamer search looking for better side chain conformations, aiming to model the expected interactions of the protein ligand with its receptors.

FoldX is able to perform amino acid mutations accommodating a new residue and its surrounding amino acids the following way: It first mutates the selected position to alanine and annotates the side chain energies of the surrounding residues. Then it mutates this alanine to the selected amino acid and re-calculates the side chain energies of the same surrounding residues. Those that exhibit an energy difference are then mutated to themselves to see if another rotamer will be more favourable. Most terms of the scoring function are balanced with respect to a reference state, to simulate the denatured protein. The side chain conformers are all compared relative to the reference state and finally candidate sequences with modelled structures (PDB coordinates) are produced. Energy evaluation of the modelled structure is performed simultaneously as part of this methodology, using the same program (FoldX, such as that available at http://foldx.crg.es). In a separate step the energy of the receptor-ligand complex and of the isolated ligand and receptor is calculated using FoldX, this allows the calculation of the free energy of interaction between ligand and receptor (in kcal mol-1). The force field module of FoldX evaluates the properties of the structure, such as its atomic contact map, the accessibility of its atoms and residues, the backbone dihedral angles, in addition to the H-bond network and electrostatic network of the protein. The contribution of water molecules making two or more H-bonds with the protein is also taken into account. FoldX then proceeds to calculate all force field components: polar and hydrophobic solvation energies, van der Waals' interactions, van der Waals' clashes, H-bond energies, electrostatics, and backbone and side chain entropies.

Using this program, all possible amino acid substitutions (preferably with the exception of Glycine, Proline, and Cysteine) are then introduced at the selected residue positions in the protein ligand in conformations (side chain rotamers) that are compatible with the rest of the structure. Glycine, Proline, and Cysteine are preferably omitted because Gly and Pro can influence the backbone conformation relatively more than other amino acids (Gly is more flexible, Pro less so). Also these residues have relatively large effects in the denatured state (Gly high entropy, Pro lower). The SH group of Cys can lead to unwanted aggregation and the like. Favourable mutations are then evaluated in terms of free energy (kcal mol-1), and unfavourable mutations (e.g. high Van der Waals' clashes) eliminated. An output of sequences and coordinates is then obtained and ranked in terms of changes in free energy, for example, using the Fold-X program. In this way, the method of this aspect of the invention substitutes one or more residues in the ligand protein substituted for replacement residues that include amino acid side-chain conformations that are predicted to fit into the binding interface with the target receptor so as to provide an increase in binding affinity for that target receptor.

Favourable mutations can be combined and evaluated in terms of changes in free energy (kcal mol-1). Unfavourable combinations (e.g. high Van der Waals' clashes) can be eliminated. If necessary, outputs of sequences and co-ordinates are, subsequent to the design process, reintroduced into the design algorithm for a further round or rounds of design. 2nd, 3rd, 4th, 5th or more rounds of design are sometimes used.

Once amino acid substitutions have been identified, mutated proteins are produced experimentally using site-directed mutagenesis, expressed, purified and then tested for stability or selectivity/specificity and for retention of biological activity. Identified molecules can be further mutated using conventional techniques of molecular evolution, to develop more specific therapeutic lead molecules. In the case of molecular evolution, one particularly useful technology is phage display.

The TRAIL variant of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature TRAIL variant may be fused with another compound, such as a compound to increase the half-life of the TRAIL variant (for example, polyethylene glycol).

These fusion proteins can be obtained by cloning a polynucleotide encoding a TRAIL variant in frame to the coding sequences for a heterologous protein sequence.

The term “heterologous”, when used herein, is intended to designate any polypeptide other than a TRAIL variant according to the invention. Examples of heterologous sequences, that can be comprised in the fusion proteins connected either at the N- or C-terminus, include: extracellular domains of membrane-bound protein, immunoglobulin constant regions (Fc regions), multimerization domains, domains of extracellular proteins, signal sequences, export sequences, tumour targeting peptides and sequences allowing purification by affinity chromatography.

Many of these heterologous sequences are commercially available in expression plasmids since these sequences are commonly included in fusion proteins in order to provide additional properties without significantly impairing the specific biological activity of the protein fused to them¹⁷. Examples of such additional properties are a longer lasting half-life in body fluids, the extracellular localization, or an easier purification procedure as allowed by the a stretch of histidines forming the so-called “histidine tag”¹⁸ or by the “HA” tag, an epitope derived from the influenza hemagglutinin protein¹⁸. If needed, the heterologous sequence can be eliminated by a proteolytic cleavage, for example by inserting a proteolytic cleavage site between the protein and the heterologous sequence, and exposing the purified fusion protein to the appropriate protease. These features are of particular importance for the fusion proteins since they facilitate their production and use in the preparation of pharmaceutical compositions. For example, the TRAIL variant may be purified by means of a hexa-histidine peptide fused at the C-terminus. When the fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of the substances of the invention and the immunoglobulin sequence. The resulting fusion protein has improved properties, such as an extended residence time in body fluids (i.e. an increased half-life), increased specific activity, increased expression level, or the purification of the fusion protein is facilitated.

In one embodiment, the protein is fused to the constant region of an Ig molecule. Examples of Ig molecules include heavy chain regions, like the CH2 and CH3 domains of human IgG1. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

In a further preferred embodiment, the TRAIL variant may comprise at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

To facilitate biological synthesis of TRAIL variants, one aspect of the invention provides nucleic acid molecules that encode the TRAIL variants listed above. The coding sequence for WT TRAIL, is given in accession number NM_{—}003810. Nucleic acid molecules coding for TRAIL variants according to the invention may be derived from this sequence by supplementing the appropriate coding sequence at the mutation point(s). Examples of preferred nucleic acid molecules according to the invention are variants of the sequences presented in SEQ ID NO: 2 (full length gene); or nucleotides 88-933 (846 nucleotides long) of SEQ ID NO:2 which is the coding sequence. A preferred coding sequence for WT rhTRAIL (amino acids 114-281) is presented in SEQ ID NO:4 (rhTRAIL 114-281 preceded by methionine) and so the variants of the present invention are preferably encoded by variants of this sequence.

In order to introduce mutation(s) into the full length or rhTRAIL coding sequence, the skilled person will be perfectly able to substitute the necessary codon at the relevant position in the sequence. All amino acid numbers referred to herein relate to the full length TRAIL protein sequence. To account for codon bias between different host organisms, the skilled person may refer to published texts on this matter or common general knowledge.

For example, codon usage in various different species can be found at: http://www.kazusa.or.jp/codon/; in particular for Escherichia coli codon usage information can be found at: http://www.kazusa.or.jp/codon/cgi-bin/showcodon.cgi?species=155864.

Additionally, the primers used to generate the various mutants described herein are illustrated in the below Table. From this, it will be perfectly possible for the skilled person to derive coding sequences for full length and soluble fragment mutants according to the invention.

| Position | Primer name | Sequence (5′-3′) |
|---|---|---|
| 218 | D218H_for | CACAAGTTATCCTCATCCTATATTGTTG (SEQ ID NO: 5) |
| 218 | D218H_rev | CAACAATATAGGATGAGGATAACTTGTG (SEQ ID NO: 6) |
| 218 | D218Y_for | CACAAGTTATCCTATCCCTATATTGTTG (SEQ ID NO: 7) |
| 218 | D218Y_rev | CAACAATATAGGGATAGGATAACTTGTG (SEQ ID NO: 8) |
| 131 | G131R_for | CTGGGACCAGACGTAGAAGCAACAC (SEQ ID NO: 9) |
| 131 | G131R_rev | GTGTTGCTTCTACGTCTGGTCCCAG (SEQ ID NO: 10) |
| 199 201 | N199H K201R_for | GGAAATAAAAGAACATACACGCAACGACAAACAAATGG (SEQ ID NO: 11) |
| 199 201 | N199H K201R_rev | CCATTTGTTTGTCGTTGCGTGTATGTTCTTTTATTTCC (SEQ ID NO: 12) |
| 199 201 | N199R K201H_for | GGAAATAAAAGAACGCACACATAACGACAAACAAATGG (SEQ ID NO: 13) |
| 199 201 | N199R K201H_rev | CCATTTGTTTGTCGTTATGTGTGCGTTCTTTTATTTCC (SEQ ID NO: 14) |
| 149 | R149M_for | AAGGCTCTGGGCATGAAAATAAACTC (SEQ ID NO: 15) |
| 149 | R149M_rev | GAGTTTATTTTCATGCCCAGAGCCTT (SEQ ID NO: 16) |
| 149 | R149N_for | AAGGCTCTGGGCAACAAAATAAACTC (SEQ ID NO: 17) |
| 149 | R149N_rev | GAGTTTATTTTGTTGCCCAGAGCCTT (SEQ ID NO: 18) |
| 149 | R149K_for | AAGGCTCTGGGCAAAAAAATAAACTC (SEQ ID NO: 19) |
| 149 | R149K_rev | GAGTTTATTTTTTTGCCCAGAGCCTT (SEQ ID NO: 20) |
| 149 | R149I_for | AAGGCTCTGGGCATTAAAATAAACTC (SEQ ID NO: 21) |
| 149 | R149I_rev | GAGTTTATTTTAATGCCCAGAGCCTT (SEQ ID NO: 22) |
| 159 | S159R_for | GGAATCATCAAGGAGAGGGCATTCATTC (SEQ ID NO: 23) |
| 159 | S159R_rev | GAATGAATGCCCTCTCCTTGATGATTCC (SEQ ID NO: 24) |
| 193 | Q193H_for | TACTTTCGATTTCACGAGGAAATAAAAG (SEQ ID NO: 25) |
| 193 | Q193H_rev | CTTTTATTTCCTCGTGAAATCGAAAGTA (SEQ ID NO: 26) |
| 193 | Q193K_for | TACTTTCGATTTAAGGAGGAAATAAAAG (SEQ ID NO: 27) |
| 193 | Q193K_rev | CTTTTATTTCCTCCTTAAATCGAAAGTA (SEQ ID NO: 28) |
| 199 | N199R_for | GGAAATAAAAGAACGCACAAAGAACGACAAACAAATGG (SEQ ID NO: 29) |
| 199 | N199R_rev | CCATTTGTTTGTCGTTCTTTGTGCGTTCTTTTATTTCC (SEQ ID NO: 30) |
| 201 | K201R_for | GGAAATAAAAGAAAACACAAGGAACGACAAACAAATGG (SEQ ID NO: 31) |
| 201 | K201R_rev | CCATTTGTTTGTCGTTCCTTGTGTTTTCTTTTATTTCC (SEQ ID NO: 32) |
| 204 | K204D_for | CACAAAGAACGACGACCAAATGGTCCAATATATT (SEQ ID NO: 33) |
| 204 | K204D_rev | AATATATTGGACCATTTGGTCGTCGTTCTTTGTG (SEQ ID NO: 34) |
| 204 | K204E_for | CACAAAGAACGACGAGCAAATGGTCCAATATATT (SEQ ID NO: 35) |
| 204 | K204E_rev | AATATATTGGACCATTTGCTCGTCGTTCTTTGTG (SEQ ID NO: 36) |
| 204 | K204L_for | CACAAAGAACGACTTGCAAATGGTCCAATATATT (SEQ ID NO: 37) |
| 204 | K204L_rev | AATATATTGGACCATTTGCAAGTCGTTCTTTGTG (SEQ ID NO: 38) |
| 204 | K204Y_for | CACAAAGAACGACTACCAAATGGTCCAATATATT (SEQ ID NO: 39) |
| 204 | K204Y_rev | AATATATTGGACCATTTGGTAGTCGTTCTTTGTG (SEQ ID NO: 40) |
| 212 | K212R_for | CCAATATATTTACCGCTACACAAGTTATCCTGACC (SEQ ID NO: 41) |
| 212 | K212R_rev | GGTCAGGATAACTTGTGTAGCGGTAAATATATTGG (SEQ ID NO: 42) |
| 215 | S215D_for | CCAATATATTTACAAATACACAGACTATCCTGACCCTAT (SEQ ID NO: 43) |
| 215 | S215D_rev | ATAGGGTCAGGATAGTCTGTGTATTTGTAAATATATTGG (SEQ ID NO: 44) |
| 215 | S215E_for | CCAATATATTTACAAATACACAGAGTATCCTGACCCTAT (SEQ ID NO: 45) |
| 215 | S215E_rev | ATAGGGTCAGGATACTCTGTGTATTTGTAAATATATTGG (SEQ ID NO: 46) |
| 215 | S215H_for | CCAATATATTTACAAATACACACACTATCCTGACCCTAT (SEQ ID NO: 47) |
| 215 | S215H_rev | ATAGGGTCAGGATAGTGTGTGTATTTGTAAATATATTGG (SEQ ID NO: 48) |
| 215 | S215K_for | CCAATATATTTACAAATACACAAAGTATCCTGACCCTAT (SEQ ID NO: 49) |
| 215 | S215K_rev | ATAGGGTCAGGATACTTTGTGTATTTGTAAATATATTGG (SEQ ID NO: 50) |
| 251 | K251D_for | GGAATATTTGAGCTTGACGAAAATGACAGAATTTTTG (SEQ ID NO: 51) |
| 251 | K251D_rev | CAAAAATTCTGTCATTTTCGTCAAGCTCAAATATTCC (SEQ ID NO: 52) |
| 251 | K251E_for | GGAATATTTGAGCTTGAGGAAAATGACAGAATTTTTG (SEQ ID NO: 53) |
| 251 | K251E_rev | CAAAAATTCTGTCATTTTCCTCAAGCTCAAATATTCC (SEQ ID NO: 54) |
| 251 | K251Q_for | GGAATATTTGAGCTTCAGGAAAATGACAGAATTTTTG (SEQ ID NO: 55) |
| 251 | K251Q_rev | CAAAAATTCTGTCATTTTCCTGAAGCTCAAATATTCC (SEQ ID NO: 56) |

The nucleic acid may be DNA or RNA (or hybrids thereof), or their analogues, such as those containing modified backbones (e.g. phosphorothioates) or peptide nucleic acids (PNA). It may be single stranded (e.g. mRNA) or double stranded, and the invention includes both individual strands of a double-stranded nucleic acid (e.g. for antisense, priming or probing purposes). It may be linear or circular. It may be labelled. It may be attached to a solid support.

Nucleic acid according to the invention can, of course, be prepared in many ways e.g. by chemical synthesis (e.g. phosphoramidite synthesis of DNA) in whole or in part, by nuclease digestion of longer molecules, by ligation of shorter molecules, from genomic or cDNA libraries, by use of nucleic acid polymerases etc.

Accordingly, the present invention also provides vectors (e.g. plasmids) comprising nucleic acid of the invention (e.g. expression vectors and cloning vectors) and host cells (prokaryotic or eukaryotic) transformed with such vectors.

The invention also provides a process for producing a TRAIL variant of the invention, comprising the step of culturing a host cell transformed with nucleic acid of the invention under conditions that induce expression of the variant.

Suitable expression systems for use in the present invention are well known to those of skill in the art and many are described in detail in Sambrook (1989)²⁰ and Fernandez et al. (1998)²¹. Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook²⁰. Generally, the encoding gene can be placed under the control of a control element such as a promoter, ribosome binding site (for bacterial expression) and, optionally, an operator, so that the DNA sequence encoding the desired peptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the peptides of the invention.

For long-term, high-yield production of a recombinant polypeptide, stable expression is preferred. For example, cell lines that stably express the peptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers et al. ²². Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; U.S. Pat. No. 5,608,143 and Zenk (1991)²³. In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred prokaryotic expression systems include those that use streptococci, staphylococci, E. coli, Streptomyces and Bacillus subtilis as host cells.

Examples of particularly suitable fungal expression systems include those that use yeast (for example, S. cerevisiae) and Aspergillus as host cells.

A further aspect of the invention may comprise a pharmaceutical composition comprising a mutant cytokine, nucleic acid or vector as described above, in conjunction with a pharmaceutically-acceptable carrier. The invention provides a pharmaceutical composition comprising (a) TRAIL variant, nucleic acid or vector as described above and (b) a pharmaceutical carrier.

Component (a) is the active ingredient in the composition, and this is present at a therapeutically effective amount e.g. an amount sufficient to induce apoptosis. The precise effective amount for a given patient will depend upon their size and health, the nature and extent of the disease, and the composition or combination of compositions selected for administration. The effective amount can be determined by routine experimentation and is within the judgement of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg, preferably about 10 mg/kg. A suitable dose should be used so as to achieve a concentration in the patient of between 0.01 ng/ml and 100 μg/ml blood, preferably between 1 ng/ml and around 1 μg/ml, more preferably around 10-100 ng/ml in human blood. TRAIL variants may be included in the composition in the form of salts and/or esters.

Carrier (b) can be any substance that does not itself induce the production of antibodies harmful to the patient receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers can include liquids such as water, saline, glycerol and ethanol. Auxiliary substances, such as wetting or emulsifying agents, pH buffering substances, and the like, can also be present in such vehicles. Liposomes are suitable carriers. A thorough discussion of pharmaceutical carriers is available in Gennaro ²⁴.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be lyophilised.

The pharmaceutical composition is preferably sterile. It is preferably pyrogen-free. It is preferably buffered e.g. at between pH 6 and pH 8, generally around pH 7.

The invention also provides a delivery device containing a pharmaceutical composition of the invention. The device may be, for example, a syringe.

TRAIL variants of the invention may be co-administered with one or more other compounds, preferably antitumour compounds, more preferably those which are active against the cancerous cells targeted by the variants of the invention and/or those which increase responsiveness of the tumour to the TRAIL variants. Compositions of the invention may thus include one or more antitumour agents, examples of which will be known to those of skill in the art and include gamma irradiation as well as chemotherapeutic drugs such as alkylating agents, anti-metabolites, plant alkaloids and terpenoids, vinca alkaloids, podophyllotoxin derivatives, taxanes, topoisomerase inhibitors, antitumour antibiotics, monoclonal antibodies, DNA damaging drugs, histone deacetylase inhibitors, proteasome inhibitors, hormones and so on. Preferably, the chemotherapeutic agent used acts to increase the surface expression of the DR4 (TRAIL-R1) receptor and/or enhance apoptosis-induction through DR4.

By “enhanced apoptosis induction” it is meant that the chemotherapeutic agent increases the number of cells that undergo apoptosis in the presence of the chemotherapeutic agent when compared to a sample which was not exposed to the chemotherapeutic agent. Preferably the increase is 1.5-fold (more preferably 2-fold, 4-fold, 8-fold, 10-fold, 20-fold, 100-fold or even 1000-fold).

The invention provides a TRAIL variant of the invention for use as a medicament. The invention also provides a method for treating a subject suffering from or at risk of contracting a disease, comprising administering to the subject a pharmaceutical composition of the invention. The invention also provides the use of a pharmaceutical composition of the invention in the manufacture of a medicament for treating a subject. Particularly suitable diseases include autoimmune disorders and cancer. Examples of autoimmune disorders where the inventors envisage potential medical use of the receptor-selective TRAIL variants are different forms of autoimmune arthritis and multiple sclerosis. Suitable cancers include leukaemia, lymphoma, melanoma, prostate, bladder, kidney, head and neck, liver and breast cancer, cancers of the lung, ovaries and colon. In a preferred embodiment the cancer is ovarian cancer, colon cancer, lymphoma (for example mantle cell lymphoma) or leukemia (for example acute lymphocytic leukemia (ALL), acute myelogenous leukemia (AML), chronic lymphocytic leukemia (CLL) and chronic myelogenous leukemia (CML)).

Preferably, suitable cancers include cells that express the DR4 receptor on the surface. Such cancer cells are readily identifiable by various means known to those of skill in the art which include, but are not limited to, immunocytochemistry with receptor specific antibodies, Fluorescent-activated cell sorting (FACS) with receptor specific antibodies, western blot analysis with receptor specific antibodies etc. DR4 specific antibodies can be obtained, for example, from Abcam (ab8414). DR5 antibodies are available as well (Sigma-Aldrich, D3938).

In certain embodiments of the invention, as described above, it is preferred that a compound according to the invention is administered in conjunction with another agent, such as an anti-tumour agent. Suitable, examples of such agents for use in combination with the pharmaceutical composition of the present invention are known in the art and examples are listed above.

It is also envisioned that a pharmaceutical composition, comprising the variant TRAIL of the invention, may on occasion be administered in conjunction with antibodies against one or more DR5, DcR1 or DcR2. In such a manner it is possible further to block signalling through the DR5 specific pathway or binding of the TRAIL variant to decoy receptors. This is advantageous since any residual binding activity to receptors other than DR4 is inhibited and specificity of the mutant cytokine of the invention is enhanced even further.

The subject is preferably a mammal, more preferably a human. The human may be an adult or a child. A composition intended for children may also be administered to adults e.g. to assess safety, dosage, immunogenicity, etc.

Compositions of the invention will generally be administered directly to a subject. Direct delivery may be accomplished by parenteral injection (e.g. subcutaneously, intraperitoneally, intravenously, intramuscularly, or to the interstitial space of a tissue; also by direct injection into the tumour).

Dosage treatment can be a single dose schedule or a multiple dose schedule.

The uses and methods of the invention can be used therapeutically (e.g. for treating an existing disease) or prophylactically (e.g. in a situation where the incidence of disease is anticipated, perhaps through genetic screening or familial history). Therapeutic use is preferred, and efficacy of treatment can be tested by monitoring cell titres after administration of the pharmaceutical composition of the invention, or by monitoring symptoms.

In a further aspect, the invention thus provides a TRAIL variant, or a nucleic acid molecule encoding such a molecule, or a vector containing a nucleic acid molecule as described, for use in the therapy or diagnosis of a disease in which cytokines are implicated. This aspect of the invention includes a method for the treatment of such a disease, comprising administering to a patient, a cytokine, nucleic acid or vector as described above, in a therapeutically-acceptable amount. Preferred patients include mammals, and are preferably humans.

TRAIL variants according to the invention may also be used in diagnosis, for example, by assessing the level of expression or activity of a gene or protein (such as an over-expressed receptor) in tissue from said patient and comparing said level of expression or activity to a control level, wherein a level that is different to said control level is indicative of disease.

TRAIL variants may also be used for assays, for example, in cell lines to test for potential anticancer activity of candidate compounds. Of particular interest are cell lines which were derived from cancerous tissue of a mammal, such as a human. Such cell lines provide useful models for the study of disease and may also be used in screening regimes for the identification of compounds that are effective in the treatment or diagnosis of disease.

The invention also provides a method for inducing apoptosis in cell lines that signal primarily through the DR4-specific pathway. Such cell lines can be easily identified by various means. For example, receptor specific neutralising antibodies can be used to inhibit signalling through one given receptor. For example, antibodies against DR4 and DR5 can be used to inhibit signalling through DR4 or DR5, respectively. Cell lines, in which a DR5-specific antibody cannot reduce TRAIL-induced apoptosis, but a DR4-specific antibody can, are good candidates for such cell lines. Reduced ability thereby refers to a reduction by 20% (more preferably 30%, 40%, 50%, 60%, 70%, 80%, 90%, 95% or 100%) of the number of cells which undergo apoptosis compared to a sample in which the DR4 antibody is not present. Further methods to identify such cells are readily available to those skilled in the art. Examples of such cell lines include, but are not limited to, ML-1, EM-2, HL-60 and MOLM-13.

A still further aspect of the invention may comprise transgenic or humanised non-human animals that have been transformed to express a mutant cytokine as described above. Such transgenic animals provide useful models for the study of disease and may also be used in screening regimes for the identification of compounds that are effective in the treatment or diagnosis of disease. Preferred such animals are rodents, particularly mice.

Various aspects and embodiments of the present invention will now be described in more detail by way of example. It will be appreciated that modification of detail may be made without departing from the scope of the invention.

### BRIEF DESCRIPTION OF DRAWINGS

FIG. 1: Results of FoldX calculation. A negative ΔΔGᵢ indicates an improvement in receptor binding and a positive ΔΔGᵢ indicates a decrease in receptor binding.

FIG. 2: Pre-steady state binding of the purified ligands WT rhTRAIL, D218H and D218Y to immobilized DR4-Ig, DR5-Ig, DcR1-Ig and DcR2-Ig receptor chimeras was assessed in real lime using surface plasma resonance (SPR). (A) Receptor binding of WT rhTRAIL, D218H and D218Y towards DR5-Ig as determined by SPR or towards DR4-Ig; (B) Receptor binding of WT rhTRAIL, D218H and D218Y towards DcR1-Ig as determined by SPR or towards DcR2-Ig. DR4-Ig and DR5-Ig preparations formulated with BSA were immobilized on the sensor surface. Binding was calculated relative to the response of WT rhTRAIL at 250 nM. (C) Pre-steady state binding of the purified ligands to immobilized OPG receptor chimera was assessed in real time using surface plasma resonance (SPR). Receptor binding of WT rhTRAIL, D218H and D218Y towards OPG was determined by SPR. OPG preparations formulated with BSA were immobilized on the sensor surface. Binding was calculated relative to the response of WT rhTRAIL at 250 nM. Competition ELISA experiments were performed using DR4-Ig (D) or soluble DR5-Ig (E) as competitor. rhTRAIL WT or variants were pre-incubated with 0-500 ng/well DR4 or DR5 during 30 min. Pre-incubated solutions were added to microtiter plates coated with DR4-Ig. Binding of the selective variants at various concentrations of soluble receptor towards the immobilized DR4-Ig was calculated relative to the value measured on the presence of 0 ng/well of soluble receptor.

FIG. 3: Biological activity of WT rhTRAIL, the D218Y variant and the DR5 selective ligand D269H/E195R in DR5 sensitive Colo205 cells (A) and in DR4 sensitive ML-1 (B) and EM-2 cells (C). Percentage apoptosis was measured as percentage Annexin V positivity after 3 hrs of incubation with 20 ng/ml (Colo205) or 100 ng/ml (EM-2) of WT rhTRAIL or variant. These concentrations were chosen as these are the concentrations were WT rhTRAIL starts to show its maximum apoptosis inducing activity. Apoptosis inducing activity is calculated relative to the apoptosis inducing activity of WT rhTRAIL at these concentrations.

FIG. 4: Table and plotted graph of the ΔΔG values (in kcal/mol) calculated for the design of 20 mutants for DR4 selectivity

FIG. 5: Pre-steady state binding of the purified ligands to immobilized DR4-Ig and DR5-Ig receptor chimeras was assessed in real time using surface plasma resonance (SPR). Receptor binding of WT rhTRAIL, G131R, N199RK201H and G131RN199RK201H towards DR5-Ig and DR4-Ig as determined by SPR. DR4-Ig and DR5-Ig preparations formulated with BSA were immobilized on the sensor surface. Binding was calculated relative to the response of WT rhTRAIL at 250 nM.

FIG. 6: Cell surface expression of TRAIL receptors in (A) ML-1, EM-2 and A2780 cells and (B) HL-60, Oci-AML3 and MOLM-13. Cells were seeded (ML-1: 300 000 cells/ml, EM-2: 300 000 cells/ml, A2780: 350 000 cells/ml, HL-60: 300 000 cells/ml, Oci-AML3: 750 000 cells/ml, MOLM-13: 1 000 000 cells/ml) 24 h before harvesting for immunofluorescent labelling of DR4, DR5, DcR1 and DcR2. Cells were removed from culture dish (A2780 cells were harvested by gentle trypsinization) and washed two times with 1% BSA in PBS then incubated in 1:100 dilution of primary antibody in 1% BSA in PBS for 40 minutes at room temperature (DR4 and DR5: neutralizing antibodies, Alexis, DcR1 and DcR2: neutralizing goat polyclonal antibodies, R&D Systems). After two rounds of washes in 1% BSA/PBS, cells were resuspended in 1:50 dilution of FITC-labelled secondary antibody (Sigma) and incubated for 40 minutes at room temperature. The excess secondary antibody was removed by one wash in 1% BSA in PBS and one wash in PBS. The cells were fixed in 1% formaldehyde in PBS before analysis by flow cytometry (FacsCalibur, Beckton Dickinson). AF: autofluorescent, IC: isotype control.

FIG. 7: Comparison of the pro-apoptotic potential of the potentially DR4-selective TRAIL mutant N199R/K201H and WT rhTRAIL. ML-1 cells were seeded at a density of 300 000 cells/ml 24 h before treatment with increasing doses of N199R/K201H or WT rhTRAIL for 24 h. The graph shows the percentage of dead cells determined by flow cytometric analysis of Annexin V positivity.

FIG. 8: Comparison of the pro-apoptotic potential of the potentially DR4-selective TRAIL mutant N199R/K201H and WT rhTRAIL. EM-2 cells were seeded at a density of 300 000 cells/ml 24 h before treatment with increasing doses of N199R/K201H or WT rhTRAIL for 24 h. The graph shows the percentage of dead cells determined by flow cytometric analysis of Annexin V positivity.

FIG. 9: Comparison of the pro-apoptotic potential of the potentially DR4-selective TRAIL mutant N199R/K201H, the DR5-selective mutant D269H/E195R and WT rhTRAIL. A2780 cells were seeded at a density of 350 000 cells/ml 24 h before treatment with increasing doses of N199R/K201H, D269H/E195R or WT rhTRAIL for 24 h. The graph shows the percentage of dead cells determined by flow cytometric analysis of Annexin V positivity.

FIG. 10: Pro-apoptotic potential of the DR4-selective TRAIL mutant N199R/K201H/G131R, WT rhTRAIL and the DR5-selective mutant D269H/E195R (E195R) in ML-1 cells. ML-1 acute myeloid leukaemia cells were seeded at a density of 300 000 cells/ml 24 h before treatment with increasing doses of N199R/K201H/G131R, WT rhTRAIL or D269H/E195R (indicated in this figure as E195R) for 24 h. The graphs show the percentage of dead cells determined by flow cytometric analysis of Annexin V positivity. (A) Comparison of the effect of WT rhTRAIL to the DR4-selective mutant N199R/K201H/G131R. (B) Comparison of the effect of WT rhTRAIL to the DR5-selective mutant D269H/E195R (E195R).

FIG. 11: Pro-apoptotic potential of the DR4-selective TRAIL mutant N199R/K201H/G131R, WT rhTRAIL and the DR5-selective mutant D269H/E195R (E195R) in EM-2 cells. EM-2 chronic myelogenous leukemia cells were seeded at a density of 300 000 cells/ml 24 h before treatment with increasing doses of N199R/K201H/G131R, WT rhTRAIL or D269H/E195R for 24 h. The graphs show the percentage of dead cells determined by flow cytometric analysis of Annexin V positivity. (A) Comparison of the effect of WT rhTRAIL to the DR4-selective mutant N199R/K™₂₀₁H/G131R. (B) Comparison of the effect of WT rhTRAIL to the DR5-selective mutant D269H/E195R (E195R).

FIG. 12: Comparison of the pro-apoptotic potential of the DR4-selective TRAIL mutant N199R/K201H/G131R, the DR5-selective mutant D269H/E195R (E195R) and WT rhTRAIL. A2780 cells were seeded at a density of 350 000 cells/ml 24 h before treatment with increasing doses of N199R/K201H/G131R, D269H/E195R or WT rhTRAIL for 24 h. The graph shows the percentage of dead cells determined by flow cytometric analysis of Annexin V staining.

FIG. 13: Pro-apoptotic potential of the DR4-selective TRAIL mutant N199R/K201H/G131R, WT rhTRAIL and the DR5-selective mutant D269H/E195R (E195R) in HL-60 cells. HL-60 (promyelocytic leukaemia cells, PML, a subtype of AML) cells were seeded at a density of 300 000 cells/ml 24 h before treatment with increasing doses of N199R/K201H/G131R, WT rhTRAIL or D269H/E195R for 24 h. The graphs show the percentage of dead cells determined by flow cytometric analysis of Annexin V positivity. (A) Comparison of the effect of WT rhTRAIL to the DR4-selective mutant N199R/K201H/G131R. (B) Comparison of the effect of WT rhTRAIL to the DR5-selective mutant D269H/E195R.

FIG. 14: Pro-apoptotic potential of the DR4-selective TRAIL mutant N199R/K201H/G131R, WT rhTRAIL and the DR5-selective mutant D269H/E195R (E195R) in Oci-AML3 cells. Oci-AML3 (AML) cells were seeded at a density of 750 000 cells/ml 24 h before treatment with increasing doses of N199R/K201H/G131R, WT rhTRAIL or D269H/E195R for 24 h. The graphs show the percentage of dead cells determined by flow cytometric analysis of Annexin V positivity. (A) Comparison of the effect of WT rhTRAIL to the DR4-selective mutant N199R/K201H/G131R. (B) Comparison of the effect of WT rhTRAIL to the DR5-selective mutant D269H/E195R.

FIG. 15: Pro-apoptotic potential of the DR4-selective TRAIL mutant N199R/K201H/G131R, WT rhTRAIL and the DR5-selective mutant D269H/E195R (E195R) in MOLM-13 cells. MOLM-13 (AML) cells were seeded at a density of 1 000 000 cells/ml 24 h before treatment with increasing doses of N199R/K201H/G131R, WT rhTRAIL or D269H/E195R for 24 h. The graphs show the percentage of dead cells determined by flow cytometric analysis of Annexin V positivity. (A) Comparison of the effect of WT rhTRAIL to the DR4-selective mutant N199R/K201H/G131R. (B) Comparison of the effect of WT rhTRAIL to the DR5-selective mutant D269H/E195R.

FIG. 16: Biological Activity of WT rhTRAIL and variant G131R/N199R/K201H in Colo205 cells responsive to both DR4 and DR5-mediated cell death. Colo205 cells were seeded in 96-well plates at a density of 400000 cells per ml in growth medium 24 h before treatment with increasing doses of N199RK201HG131R or WT rhTRAIL for 24 h and cell growth was measured by MTT Assay.

FIG. 17: Comparison of the pro-apoptotic potential of the potentially DR4-selective TRAIL mutant G131R/D218H and WT rhTRAIL. ML-1 cells were seeded at a density of 300 000 cells/ml 24 h before treatment with increasing doses of G131R/D218H or WT rhTRAIL for 24 h. The graph shows the percentage of dead cells determined by flow cytometric analysis of Annexin V positivity.

FIG. 18: Comparison of the pro-apoptotic potential of the potentially DR4-selective TRAIL mutant G131R/D218H and WT rhTRAIL. EM-2 cells were seeded at a density of 300 000 cells/ml 24 h before treatment with increasing doses of G131R/D218H or WT rhTRAIL for 24 h. The graph shows the percentage of dead cells determined by flow cytometric analysis of Annexin V staining.

FIG. 19: Comparison of the pro-apoptotic potential of the potentially DR4-selective TRAIL mutant G131R/D218H, the DR5-selective mutant D269H/E195R and WT rhTRAIL. A2780 cells were seeded at a density of 350 000 cells/ml 24 h before treatment with increasing doses of G131R/D218H, D269H/E195R or WT rhTRAIL for 24 h. The graph shows the percentage of dead cells determined by flow cytometric analysis of Annexin V staining.

FIG. 20: Biological Activity of WT rhTRAIL and variant G131R/N199R/K201H in BJAB cells responsive to both DR4 and DR5-mediated cell death (BJAB^{wt}) (A) and BJAB cells deficient for DR5 (BJAB^{DR5DEF}) (B). BJAB cells were seeded in 96-well plates at a density of 400000 cells per ml in growth medium+1.0 μg/ml cycloheximide with increasing doses of G131RN199RK201H or WT rhTRAIL for 24 h. Cell growth was measured by MIT Assay.

FIG. 21: Biological Activity of WT rhTRAIL and variant G131R in BJAB cells responsive to both DR4 and DR5-mediated cell death (BJAB^{wt}) (A) and BJAB cells deficient for DR5 (BJAB^{DR5 DEF}) (B). BJAB cells were seeded in 96-well plates at a density of 200000 cells per ml in growth medium+1.0 μg/ml cycloheximide with increasing doses of G131R or WT rhTRAIL for 24 h. Cell growth was measured by MIT Assay.

FIG. 22: Competitive ELISA with variant G131R/N199R/K201H in comparison with WT rhTRAIL using as competitor soluble DR5-Ig (A), soluble DR4-Ig (B), soluble DcR1-Ig (C), or soluble DcR2-Ig (D). The percentage bound to immobilized DR4-Ig is calculated relative to the amount bound at 0 ng/well of soluble competitor. WT rhTRAIL and variant were pre-incubated with 0-500 ng/well DR4 and DR5 during 30 min. Pre-incubated solutions were added to micro-titer plates coated with DR4-Ig.

FIGS. 23 (A) & (B): Variant G131R/N199R/K201H activates the death-inducing TRAIL receptors more efficiently than WT rhTRAIL in HL-60 cells. Apoptosis was measured by determination of mitochondrial membrane potential (A) or by determination of pro-caspase 8 activation (B, upper panel) The upper band is pro-caspase-8 (55/53 kDa), while the lower double band is processed caspase-8 (43/41 kDa). The lower panel shows the expression of beta-actin as an indicator of equal protein loading. (B).

FIG. 24. Inhibition of NF-κB activity with the Inhibitor κ-B kinase (IKK) inhibitor BMS-345541 (Calbiochem) enhanced the biological activity of both WT rhTRAIL and G131R/N199R/K201H. HL-60 cells were treated with 100 ng/ml (A) or 10 ng/ml (B) of WT rhTRAIL or G131R/N199R/K201H in the presence or absence of the indicated concentrations of BMS-345541 for 12 h. Induction of cell death was determined by measuring the loss of mitochondrial membrane potential with TMRE. The graphs show average percentage of cells with blow mitochondrial membrane potential (ΔΨm).

FIG. 25: Pre-steady state binding of purified TRAIL variant R149I to immobilized DR4-Ig and DR5-Ig receptor chimeras was assessed in real time using surface plasma resonance (SPR). DR4-Ig and DR5-Ig preparations were directly immobilized on the CM5 sensor surface at a density of ca. 700 RU's. Binding was calculated relative to the response of WT rhTRAIL at 250 nM.

FIG. 26: Pre-steady state binding of purified TRAIL variants S159R and K201R to immobilized DR4-Ig and DR5-Ig receptor chimeras was assessed in real time using surface plasma resonance (SPR). DR4-Ig and DR5-Ig preparations were directly immobilized on the CM5 sensor surface at a density of ca. 700 RU's. Binding was calculated relative to the response of WT rhTRAIL at 250 nM.

FIG. 27: Sensorgrams of the binding of variant S159R and WT rhTRAIL to directly immobilised DR4-Ig (A) or DR5-Ig (B).

FIG. 28: Competitive ELISA experiment with WT rhTRAIL and S159R using soluble DR4-Ig (A), soluble DR5-Ig (B), soluble DcR1-Ig (C), or soluble DcR2-Ig (D) as competitor.

FIG. 29: Pre-steady state binding of purified TRAIL variant K204Y to immobilized DR4-Ig and DR5-Ig receptor chimeras was assessed in real time using surface plasma resonance (SPR). DR4-Ig and DR5-Ig preparations were directly immobilized on the CM5 sensor surface at a density of ca. 800 RU's. Binding was calculated relative to the response of WT rhTRAIL at 250 nM.

FIG. 30: Pre-steady state binding of purified TRAIL variant S215D to immobilized DR4-Ig and DR5-Ig receptor chimeras was assessed in real time using surface plasma resonance (SPR). DR4-Ig and DR5-Ig preparations were directly immobilized on the CM5 sensor surface at a density of ca. 800 RU's. Binding was calculated relative to the response of WT rhTRAIL at 250 nM. DHER: D269H/E195R (DR5-selective rhTRAIL variant, used as a positive control for DR5 binding)

FIG. 31: Competitive ELISA with variant S215D and WT rhTRAIL (A) Competitive ELISA using soluble DR4-Ig as competitor. (B) soluble DR5-Ig as competitor. Percentage bound ligand to immobilized DR4-Ig is calculated relative to the amount bound at 0 ng/well of soluble competitor. WT rhTRAIL and variant were pre-incubated with 0-500 ng/well DR4 and DR5 during 30 min. Pre-incubated solutions were added to microtitre plates coated with DR4-Ig.

FIG. 32: Cytotoxic effect of WT rhTRAIL and variant S159R on the myeloid ML-1 (A) and EM-2 (B) cell line. ML-1 and EM-2 cells were treated with the indicated concentrations of WETrhTRAIL or variant S159R for 24 h when induction of cell death was quantified with Annexin V assay. The graphs show average percentage of Annexin V positivity induced as a measure of cell death.

FIG. 33: Cytotoxic effect of WT rhTRAIL and variant S159R on the colon carcinoma SW948 cell line.

FIGS. 34 (A), (B) & (C): Cytotoxic effect of S159R, compared to WT rhTRAIL and D269HE195R (DR5-selective variant, E195R series on graphs) in, WT HCT116 (upper panel) and p53 deficient (p53^{-/-}) HCT116 (lower panel) human colon carcinoma cell lines. The cytotoxic effect was assessed by measuring cell viability after 24 (A), 48 (B) and 72 hours (C) incubation as a function of the concentration of protein. The graph shows average viability relative to the control.

FIGS. 35 (A), (B) & (C): biological effect of TRAIL variant S159R, compared to WT rhTRAIL and D269HE195R (DR5-selective variant, E195R series on graphs), on the human pancreatic carcinoma cell line, BxPc. The biological effect was assessed by measuring cell viability after 24 (A), 48 (B) and 72 hours (C) incubation as a function of the concentration of protein.

FIGS. 36 (A), (B) & (C): biological effect of TRAIL variant S159R, compared to WT rhTRAIL and D269HE195R (DR5-selective variant, E195R series on graphs), on the human hepatocellular carcinoma HepG2 cell line. The biological effect was assessed by measuring cell viability after 24 (A), 48 (B) and 72 hours (C) incubation as a function of the concentration of protein.

FIG. 37: Biological activity of WT rhTRAIL and variants R149I and S215D on EM-2 (A) and ML-1 (B) cells as determined with Annexin V staining after 24 h treatment using flow cytometry. The Graph shows percentage of apoptotic cells.

FIG. 38: Receptor binding of rhTRAIL WT and DR4-selective mutants as determined by SPR and competitive ELISA. Receptor binding of rhTRAIL WT, D218H and D218Y to DR4-Ig was determined by SPR (A) or to DR5-Ig (B). To obtain pre-steady state data that represent proper high affinity complex formation and assuming the initial fast off-rate to represent lower complexes, the response at each concentration was recorded 30 s after the end of the injections. Receptor binding was calculated relative to the response of rhTRAIL WT at 250 nm. Competition ELISA used DR4-Ig as competitor (C) or soluble DR5-Ig as competitor (D). rhTRAIL WT or variants were pre-incubated with 0-500 ng/well DR4 or DR5 during 30 mins. Preincubated solutions were added to microtitre plates coated with DR4-Ig. Binding of the selective variants at various concentrations of soluble receptor toward the immobilised DR4-Ig was calculated to the value measured on the presence of 0 ng/well of soluble receptor.

FIG. 39: Binding energy predictions for Gly131 mutants. Predicted difference in binding energy (ΔG) of Gly-131 variants binding to different death receptors when compared with wild-type rhTRAIL as determined by FoldX version 2.8. The change in energy is measured in kcal/mol and applies to the change of a single binding interface bound to a single receptor. A negative ΔΔGᵢ indicates an improvement in receptor binding, and a positive ΔΔGᵢ indicates a decrease in receptor binding.

### EXAMPLES

### Example 1

### Expression and Purification of WT rh TRAIL and Mutants

The WT rhTRAIL and TRAIL mutant constructs were transformed to Escherichia Coli BL21 (DE3) (Invitrogen). WT rhTRAIL and mutants were grown at a 5 l batch scale in a 7.5 l fermentor (Applicon) using 2×LB medium, 1% (w/v) glucose, 100 μg/ml ampicillin and additional trace elements. The culture was grown to mid-log phase at 37° C., 30% oxygen saturation and subsequently induced with 0.1-1 mM IPTG. ZnSO4 was added at a concentration of 100 μM to promote trimer formation. Temperature was lowered to 17-28° C. and the culture was grown until stationary phase. Alternatively proteins were grown in shake flasks at a 11 scale at 250 rpm, using a similar protocol. Protein expression was induced when the culture reached OD600 0.5 and induction was continued for 5 to 16 hours. In this case, the medium used was 2×LB without additional trace elements.

The isolated pellet was resuspended in 3 volumes extraction buffer (PBS pH 8, 10% (v/v) glycerol, 7 mM β-mercapto-ethanol). Cells were disrupted using sonication and extracts were clarified by centrifugation at 40,000 g. Subsequently, the supernatant was loaded on a nickel charged IMAC Sepharose fast-flow column and WT rhTRAIL and TRAIL mutants were purified as described by Hymowitz25 with the following modifications: 10% (v/v) glycerol and a minimal concentration of 100 mM NaCl were used in all buffers. This prevented aggregation during purification. After the IMAC fractionation step, 20 μM ZnSO4 and 5 mM of DTT (instead of β-mercapto-ethanol) was added in all buffers. Finally, a gelfiltration step, using a Hiload Superdex 75 column, was included. Purified proteins were more than 98% pure as determined using a colloidal Coomassie brilliant blue stained SDS-PAGE gel. Purified protein solutions were flash frozen in liquid nitrogen and stored at −80° C.

### Example 2

### Selectivity Design

The receptor binding interface of WT TRAIL was screened for single amino acid substitutions increasing affinity for the DR4 receptor (=decreasing interaction energy (ΔΔGᵢ)) or decreasing affinity for DR5. For the TRAIL-DR5-receptor complex a crystal structure was used and for the TRAIL-DR4-receptor complex a homology model consisting of TRAIL in complex with cysteine rich domains (CRDs) 2 and 3 of DR4 was constructed based on the TRAIL-DR5-receptor complex. The CRDs 2 and 3 of DR4 show a considerable degree of sequence identity with DR5 (˜50%) and the alignment contains no insertions or deletions (not shown), consequently a modelling approach was chosen that would yield a TRAIL-DR4 model with an identical amino acid backbone conformation as the TRAIL-DR5 structure. Two different methods were used to construct TRAIL-DR4 models: 1) The WhatIf web-interface (http://swift.cmbi.ru.nl/servers/html/index.html) was used to generate an initial model of the TRAIL-DR4 receptor complex and this model was subsequently optimized by FoldX or 2) The protein design functionality of FoldX was used to mutate the DR5 sequence into the DR4 sequence and the surrounding TRAIL and receptor residues were simultaneously optimised in order to accommodate the mutated residue. The latter approach (approach 2) was found to be the preferred one.

Electrostatic charges mapped on the solvent accessible surface of TRAIL, DR5 and the DR4 model show that the surface electrostatics of TRAIL and DR5 are more complementary with each other than the surface electrostatics of TRAIL and DR4. The predictions of the energy change in the complex formation correlates with the changes in the dissociation constants measured. This implies that our method can reliably predict mutations at residue positions located at the receptor binding interface that will most severely affect the complex formation.

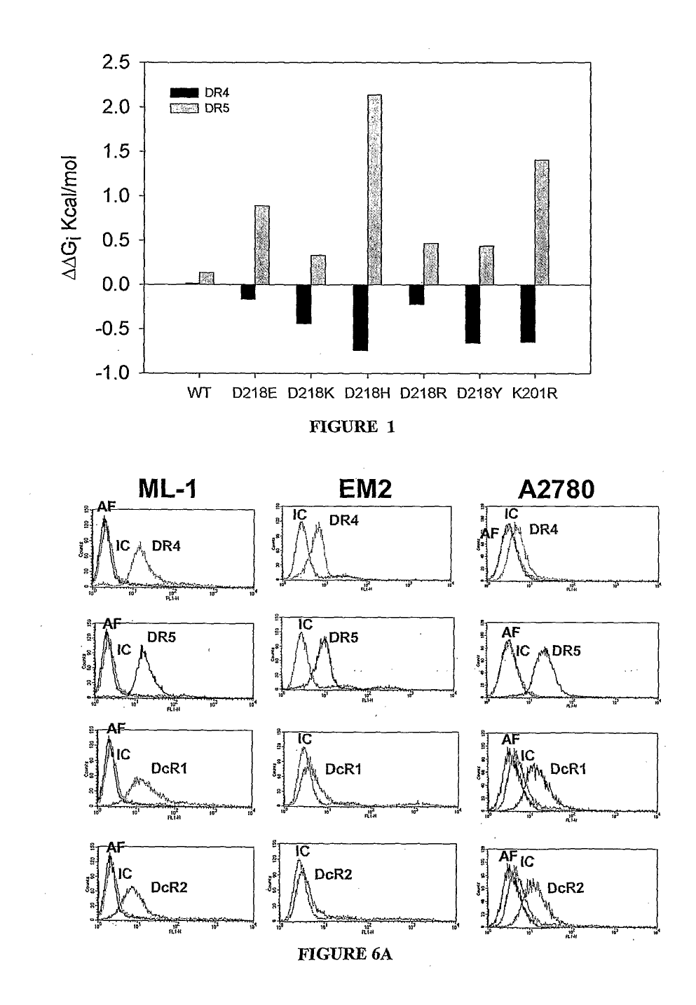

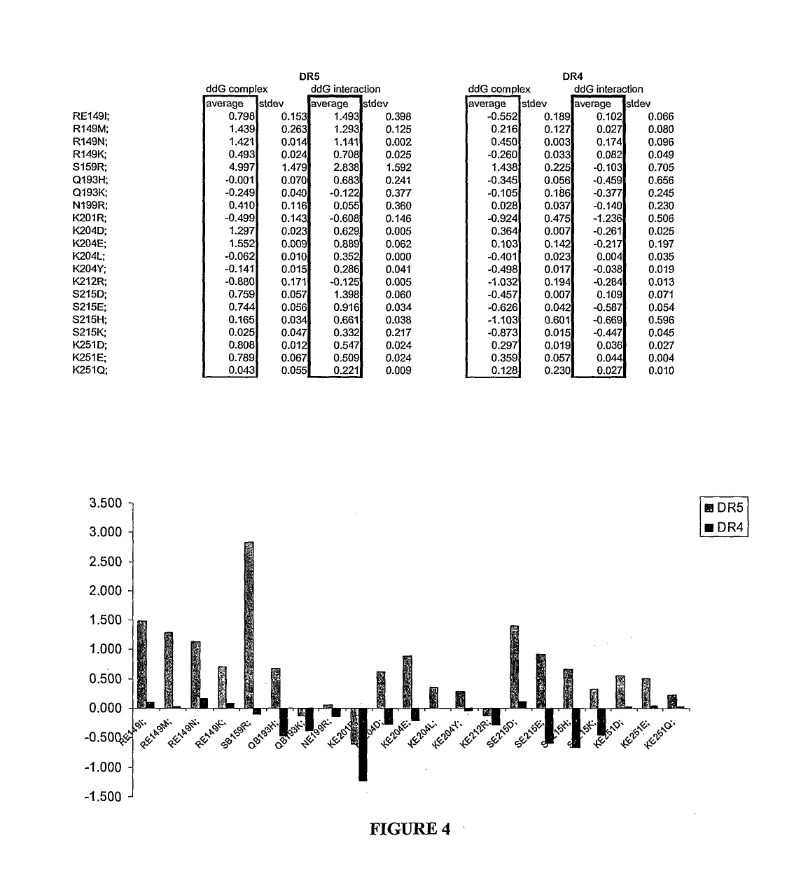

The FoldX design process proposed several TRAIL receptor interface positions and (single) amino-acid substitutions important for obtaining DR4 selectivity (FIGS. 1 and 4).

Furthermore, the following methodology was employed to select DR4 specific TRAIL mutants. A new version of the FoldX protein design algorithm was extensively validated against available experimental receptor binding data of TRAIL with DR5 in order to allow for an as accurate as possible design process. Using the thus validated FoldX design algorithm, a new model of the TRAIL-DR4 receptor complex was constructed based on the available TRAIL-DR5 structure obtained by X-ray crystallography (1D4V.pdb) by (in silico) mutating DR5 amino acids into the corresponding DR4 amino acids and subsequent optimization of the interactions of the changed amino acid residue with surrounding receptor and TRAIL residues using the FoldX design algorithm. This process was repeated using another available crystal structure of TRAIL in complex with DR5 (1D0G.pdb) as template for modelling, visual inspection of both templates revealed some structural deviations in several regions in the receptor binding interface. As the latter structure has a lower resolution and some parts of the structure are not well resolved models based on 1D0G were not further used. The TRAIL-DR4 models were subsequently validated against available experimental receptor binding data, with the model of the TRAIL-DR4 complex a correlation coefficient of R ˜0.7 could be obtained when comparing predicted and experimental alanine scan data.

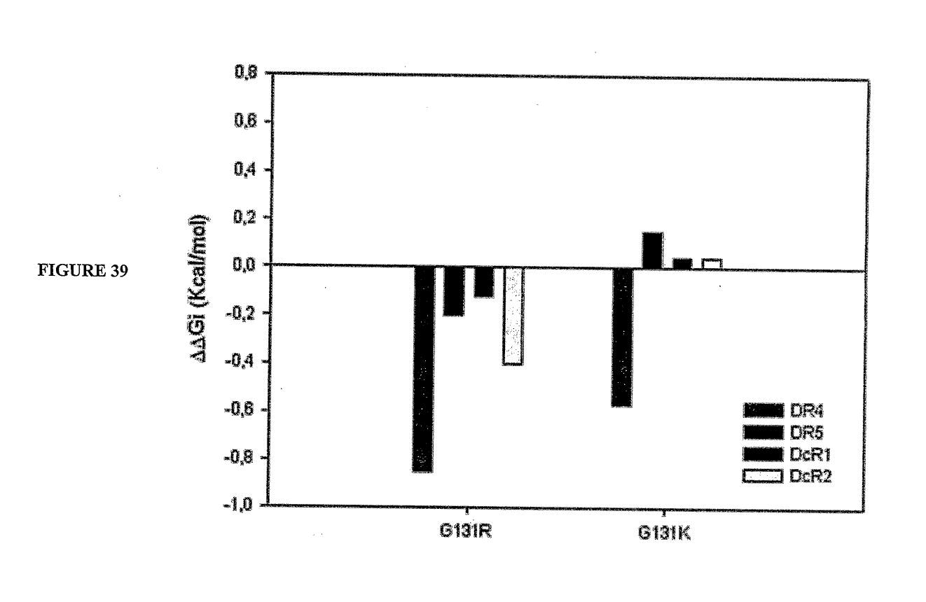

Subsequently, the design algorithm was used to perform a saturation scan (also know as a “matrix scan”) by in silico mutating each TRAIL amino acid residue located in the receptor binding interface to all 20 different natural occurring amino acids. The set of “interface” residues also included residues around (up to ˜15 Å) the receptor binding interface in order to detect mutants that would exert a receptor selective effect due to more favourable electrostatics or due to indirect effects. For each mutant, both the interaction energy and the complex stability energy were calculated for both the DR4 and the DR5 receptor. Mutants having a favourable interaction energy and complex stability energy for the DR4 receptor and an unfavourable interaction- and/or complex stability energies DR5 receptors were selected. A DR4 selective variant was defined as follows: the calculated ΔΔG interaction energy for the DR4 receptor should be less than the ΔΔG interaction energy for the DR5 receptor and the ΔΔG interaction energy as calculated for the DR4 should be less than 0.5 kcal/mol. In addition, the ΔΔG complex stability energy should be less than 0.5 kcal/mol for the TRAIL-DR4 complex and intra-chain clashes should also be less than 0.5 kcal/mol for the TRAIL-DR4 complex. The intra-chain clashes term is important when redesigning interfaces of protein complexes since it is possible that a residue has a very good interaction with the neighbour chain, but is in a very strained conformation with respect to its own chain. Thus such a conformation (having an intra-chain clash energy of more than 0.5 kcal/mol) is not realistic. For a subset of mutations another criterion was used: when mutants did not show a difference in the calculated ΔΔG interaction energy for DR4 and DR5 but having a lower calculated ΔΔG complex stability energy for the TRAIL-DR4 complex than for the TRAIL-DR5 complex and this difference in ΔΔG complex stability energy was larger than 0.7 kcal/mol, in addition to having for TRAIL-DR4 complex a ΔΔG complex stability energy and intra-chain clashes of both less than 0.5 kcal/mol, were also considered as DR4 selective mutations. Selection of mutations by both visual inspection of generated mutant structures and the above mentioned numerical values for interaction, stability and clashes resulted in table 3 describing positions and mutations having a favourable effect on selectivity of TRAIL for the DR4 receptor. FIGS. 4 and 39 represents the most interesting mutations with their calculated ddG values according to FoldX version 2.7 and 2.8. These values show a slightly improved accuracy when compared to the previous version 1.0 of FoldX used to calculate the values used in FIG. 1.

### Example 3

### Superior DR4 Affinity and Selectivity of Mutants D218Y and D218H

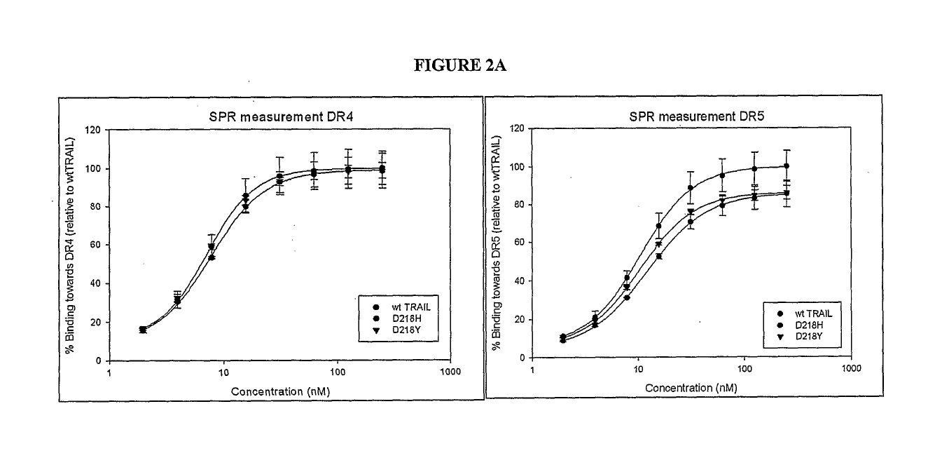

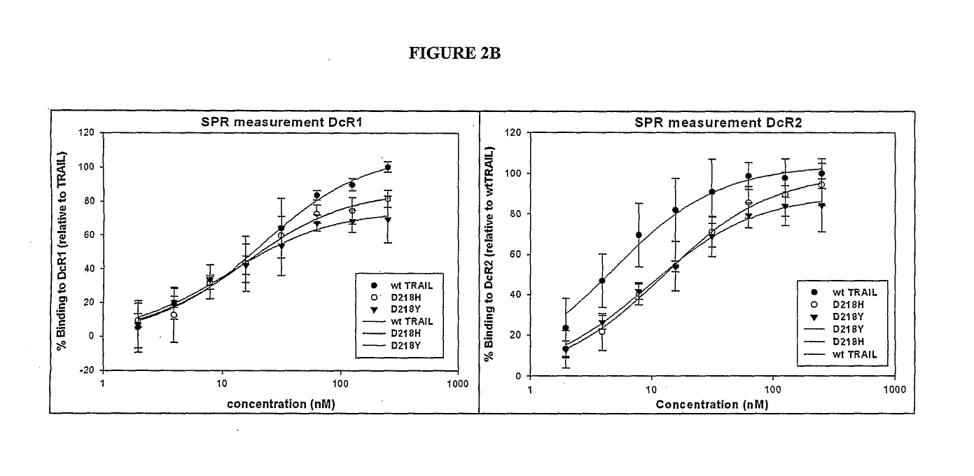

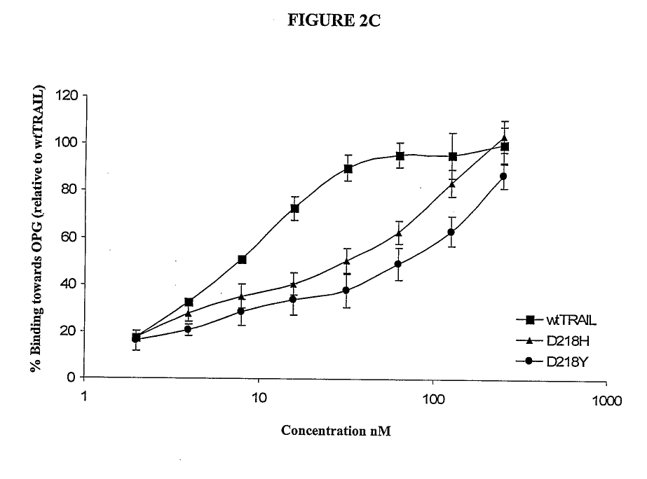

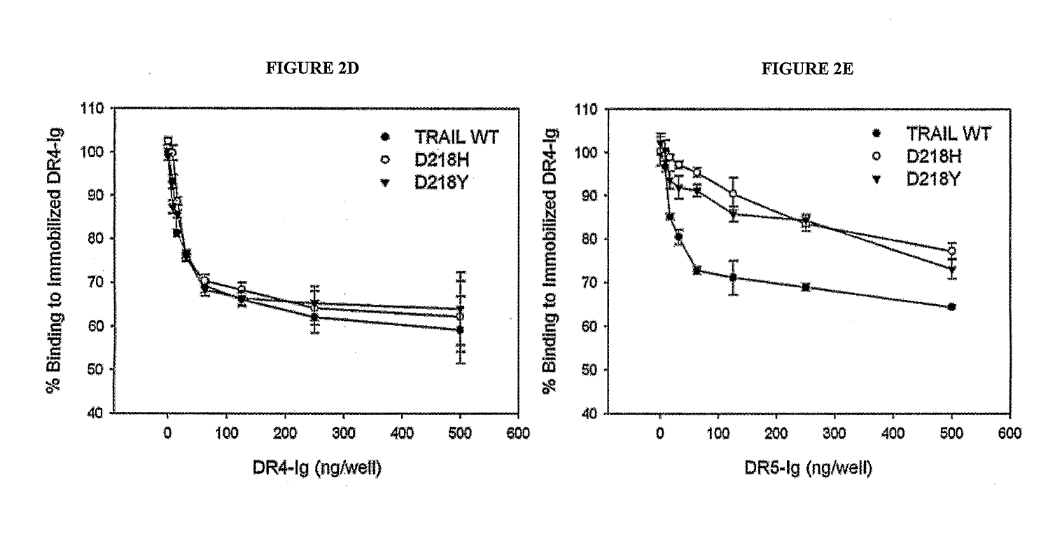

Binding of the purified ligands to immobilized DR4 and DR5 Ig receptor chimeras was assessed in real-time using Surface Plasmon Resonance (SPR). Receptor binding curves were recorded ranging from 0.5 nm to 250 nm at 37° C. The binding of D218Y and D218H towards immobilized DR5-Ig was 1.5 fold decreased when compared to WT rhTRAIL. In contrast, binding to immobilized DR4-Ig remained almost unchanged (FIG. 2A). These results show that the D218Y and 0218H variants have become DR4 selective compared to WT rhTRAIL. Interestingly, it has been reported that the affinity between WT rhTRAIL and especially DR4 is strongly dependent on temperature. However, no significant changes in binding to DR4 or DR5 were observed for WT rhTRAIL, D218H and D218Y when repeating the assay at a temperature of 25° C. (not shown). Both variants show a ˜3 fold reduction in binding to DcR2 while binding to DcR1 remains almost unchanged (˜80% of wt level at 250 nm) (FIG. 2B). Visual inspection of the sensorgrams recorded at 37° C. for DR4 and DR5 revealed that WT rhTRAIL virtually lacked an off-rate at both receptors while both D218H and D218Y showed an initial increased off-rate at both receptors. However, the off-rate of D218H and D218Y at DR4 is much smaller than the off-rate at the DR5 receptor. In total, in addition to a higher affinity, the dynamics of the DR4 complex formation are also favourable for these variants. Furthermore these mutants also show reduced binding to the OPG receptor (FIG. 2C).

To assess the selectivity of D218H and D218Y towards the DR4 receptor in the presence of the DR5 receptor a competitive ELISA experiment was performed. Soluble DR5-Ig was more than 9 fold less efficient in achieving a 50% reduction in binding towards immobilized DR4 for the D218 variants than for rhTRAIL WT (FIG. 2D, E) while soluble DR4-Ig was equally efficient in reducing the binding of rhTRAIL WT and the D218 variants towards immobilized DR4-Ig. These results indicate that D218H and D218Y preferentially bind to the DR4 receptor when both DR4 and DR5 receptors are present.

In conclusion this demonstrates that the DR4 specific mutants D218Y and D218H have an increased affinity to DR4 and decreased affinity to DR5 and DcR1, DcR2 and OPG.

### Example 4

### Biological Activity of Mutants D218Y and D218H

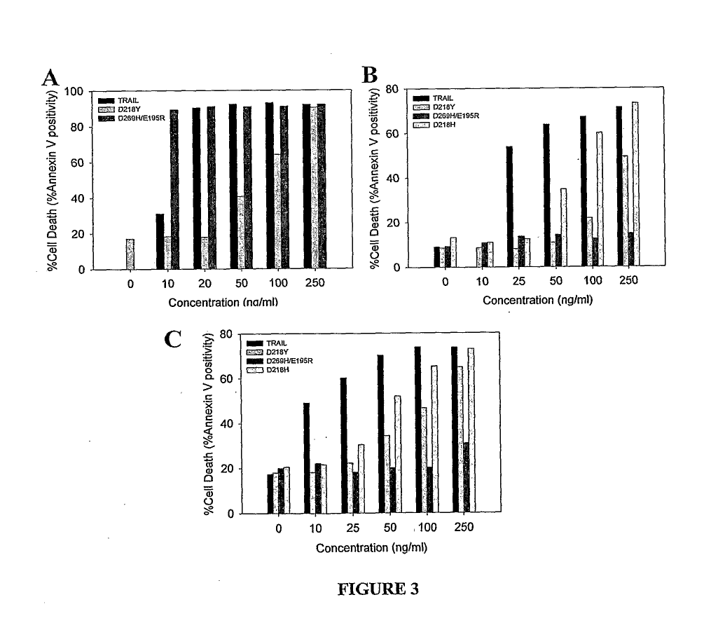

To test the biological activity of 0218Y and D218H variants, Colo205 and EM-2 cells were treated with these variants. Previously, it was established that Colo205 cells are sensitive towards TRAIL-induced apoptosis primarily mediated by DR5 and ML-1 cells, in contrast, are mainly sensitive towards TRAIL-induced apoptosis mediated by DR4. The EM-2 cell line expresses only the DR4 receptor and hence is only sensitive towards TRAIL-induced apoptosis mediated by the DR4 receptor. In Colo205 a large decrease in apoptosis inducing activity of D218Y and 0218H was observed when compared to WT rhTRAIL and the DR5 selective D269HE195R variant (FIG. 3a). In contrast, in cell lines EM-2 and ML-1, D218Y and D218H variants were able to efficiently induce apoptosis at concentrations above 100 ng/ml (FIGS. 3B and C) whereas the DR5 selective variant D269HE195R essentially lacks apoptosis inducing activity. The D218H mutant showed enhanced apoptosis inducing activity when compared to D218Y but both variants are considerably less active than WT rhTRAIL. With regard to potential DR4 selective behaviour it is important to correct for WT rhTRAIL sensitivity and to determine at what concentration the variants in comparison with WT rhTRAIL start to induce apoptosis (FIG. 3). In Colo205 cells WT rhTRAIL induces a maximum level of apoptosis at a protein concentration of ˜20 ng/ml. In ML-1 and EM-2 cells WT rhTRAIL induces a maximum level of apoptosis at a concentration of ˜100 ng/ml. While in Colo205 cells no significant apoptosis is induced by the D218Y variant at a concentration of 20 ng/ml, the D218Y variant is able to induce a significant amount of apoptosis in EM-2 cells and in ML-1 cells at a concentration of 100 ng/ml. This is illustrated in FIG. 3 for WT rhTRAIL, D218Y and the DR5 selective variant D269H/E195R. Whereas the D218Y ligand is able to efficiently induce apoptosis in EM-2 cells, it shows a marked decrease in biological activity in Colo205 cells (FIG. 3). Taken together, these results indicate that the D218Y and D218H variants induce apoptosis preferentially via DR4.

### Example 5

### Superior DR4 Affinity and Selectivity of Mutants G131R, N199RK201H and G131RN199RK201H

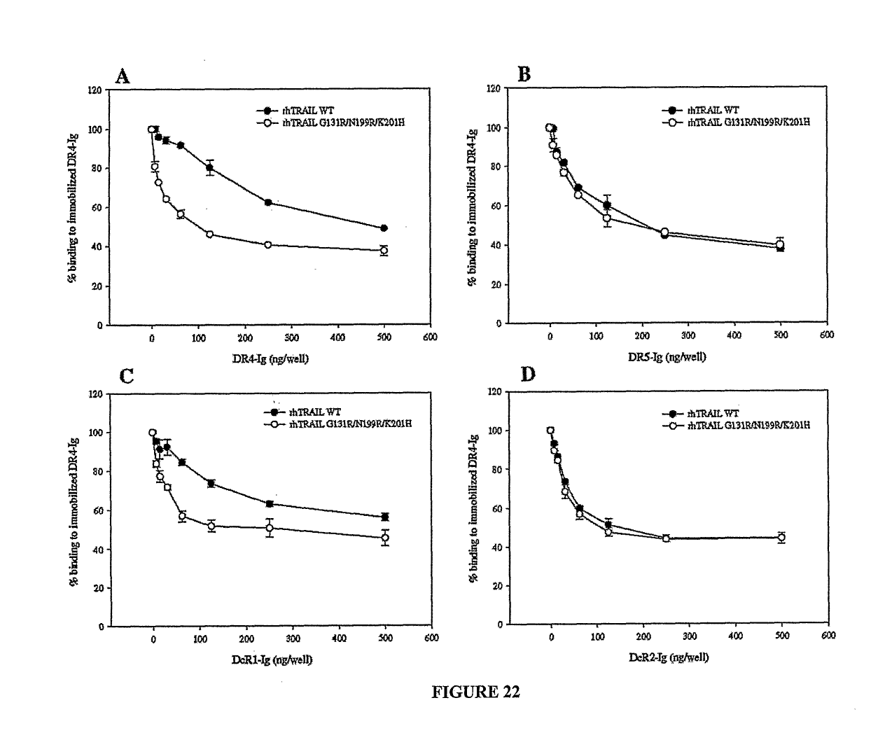

Binding of the purified ligands to immobilized DR4 and DR5 Ig receptor chimeras was assessed in real-time using surface plasmon resonance (SPR). Receptor binding curves were recorded ranging from 0.5 nm to 250 nm at 37° C. DR4-Ig and DR5-Ig preparations formulated with BSA were immobilized on the sensor surface. Binding was calculated relative to the response of WT rhTRAIL at 250 nM. The pre-steady binding assay indicates that the affinity of TRAIL variants G131R and G131R/N199R/K201H are increased in comparison to WT rhTRAIL. In comparison, the increased binding to DR5 was much less evident (FIG. 5). Competitive ELISA showed that DR4-Ig is capable to compete for binding of variant G131R/N199R/K201H to immobilised DR4-Ig much better than observed with WT, whereas DR5-Ig does not differ in competitive behaviour in presence of the variant or WT (FIGS. 22 A and B, respectively). This clearly demonstrates the increased selectivity of variant G131R/N199R/K201H for DR4.

In summary, these data indicate that these mutants selectively recognize the DR4 receptor over the DR5 receptor.

### Example 6

### Biological Activity of Mutants G131RD218H, N199RK201H and G131RN199RK201H

The potential of these mutants to induce apoptosis was tested in the cell lines ML1 (acute myeloid leukemia cell line), EM-2 (chronic myeloid leukemia cell line), A2780 (ovarian cancer cell line), HL-60 (acute myeloid leukemia cell line), OCI-AML3 (acute myeloid leukemia cell line), MOLM 13 (acute myeloid leukemia cell line) and Colo205 (colon cancer cell line).

HL60 (DSMZ, ACC 3), EM-2 (DSMZ, ACC 135) and A2780 cells (a kind gift from Dr. Steven de Jong, University Medical Centre Groningen, The Netherlands) were maintained in RPMI medium supplemented with 10% foetal bovine serum (FBS), 2 mM glutamine, 50 U penicillin and 50 mg/ml streptomycin. Cells were seeded at 500,000, 300,000 and 350,000 cells/ml respectively 24 h prior to treatment. The ML-1 cell line was maintained in RPMI medium supplemented with 20% FBS, 2 mM glutamine, 50 U/ml penicillin and 50 mg/ml streptomycin and seeded at 300,000 cells/ml 24 h prior to treatment. MOLM-13 cells (DSMZ, ACC 554) were maintained in RPMI medium supplemented with 10% FBS, 2 mM glutamine, 1 mM sodium pyruvate, 1% non essential amino acids (NEA), 50 U/ml penicillin and 50 mg/ml streptomycin and seeded at 1,000,000 cells/ml 24 h prior to treatment. OCI-AML3 cells (DSMZ, ACC 582) were maintained in MEM supplemented with 20% FBS, 2 mM glutamine, 1 mM sodium pyruvate, 1% NEA, 50 U penicillin and 50 mg/ml streptomycin and seeded at 750,000 cells/ml 24 h prior to treatment. All cells were cultured at 37° C. with 5% CO₂ in a humidified incubator. All reagents were from Sigma-Aldrich unless otherwise stated. To examine the pro-apoptotic potential of the DR4-selective TRAIL variants as a measure of their receptor selectivity and/or biological activity, cells were treated with increasing concentrations (0-250 μg/ml) of N199R/K201H, G131R/D218H, N199R/K201H/G131R, D269H/E195R or WT rhTRAIL (Triskel Therapeutics Ltd.).

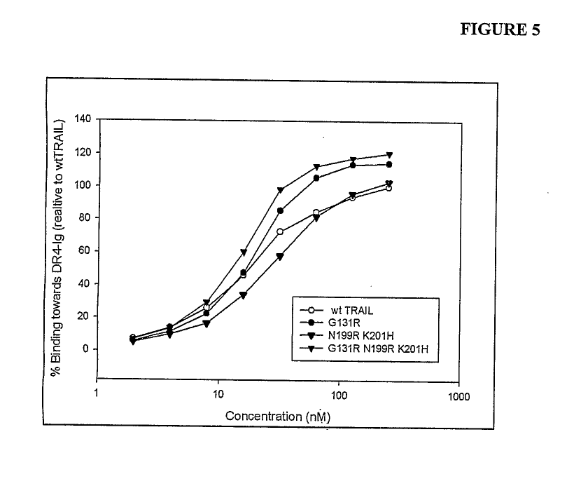

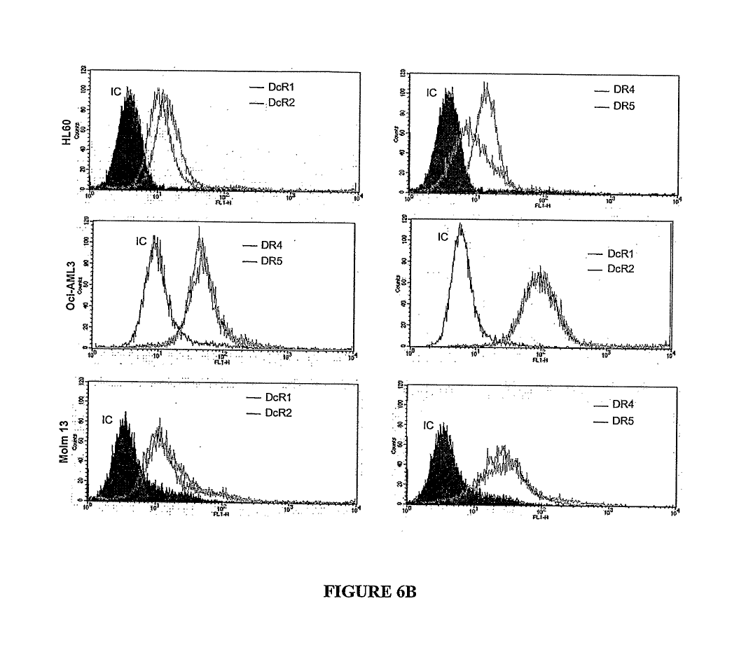

Initially it was tested if and to what extent these cell lines express the DR4 receptor on their surface. This was examined by immunofluorescently labelling of DR4, DR5, DcR1 and DcR2. Cells were removed from culture dishes; A2780 cells were harvested by gentle trypsinisation, and washed twice with 1% BSA in PBS. Cells were incubated with 1:100 dilution of primary antibodies (DR4 and DR5: neutralizing mouse monoclonal antibodies, Alexis, DcR1 and DcR2: neutralizing goat polyclonal antibodies, R&D Systems) in 1% BSA in PBS for 40 minutes at room temperature. After two washes in 1% BSA/PBS, cells were resuspended in 1:50 dilution of FITC-labelled secondary antibody and incubated for 40 minutes at room temperature. Excess secondary antibody was removed by washing first in 1% BSA in PBS and then PBS. Cells were fixed in 1% formaldehyde/PBS before analysis by flow cytometry (FacsCalibur, Beckton Dickinson). The results are shown in FIG. 6.

ML-1 and EM-2 cells express both of the death-inducing receptors (DR4 and D5). ML-1 cells also express the two decoy receptors (DcR1 and DcR2), while EM-2 cells do not, or below the limit of detectability. The cell lines HL-60, Oci-AML3 and MOLM-13 expressed all four tested receptors on their surface.

The mutant TRAIL proteins were then used in an apoptosis assay in order to evaluate their ability to induce apoptosis in the various cancer cell lines mentioned above. To this end the Annexin V assay was used.

Externalisation of phosphatidylserine (PS) to the outer leaflet of the plasma membrane is necessary for clearance of apoptotic remnants. Annexin-V, a Ca²⁺ dependent phospholipid binding protein, binds with high affinity to PS. Following treatment, cells were collected by gentle trypsinisation, pelleted by centrifugation at 350×g, washed once in ice cold Ca²⁺ buffer (10 mM HEPES/NaOH, pH 7.4, 140 mM NaCl and 2.5 mM CaCl₂) and incubated with annexin-V-FITC (IQ Corporation) for 15 min on ice in the dark. A wash step in Ca²⁺ buffer was carried out prior to acquisition on a FACSCalibur flow cytometer (Becton Dickinson). Analysis was performed using Cell Quest software (Becton Dickinson).

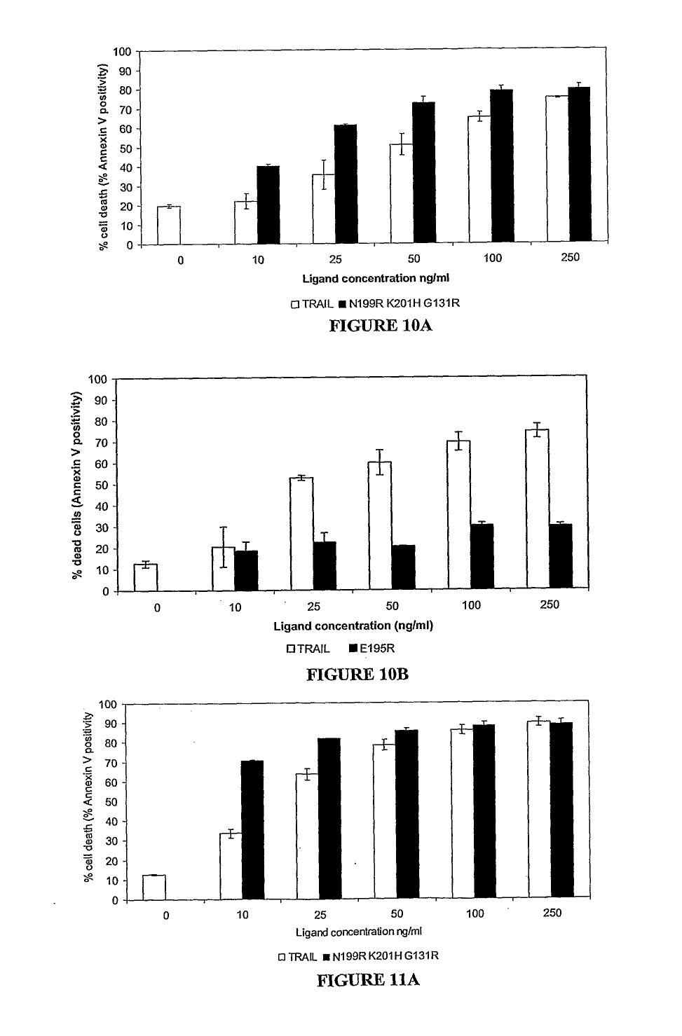

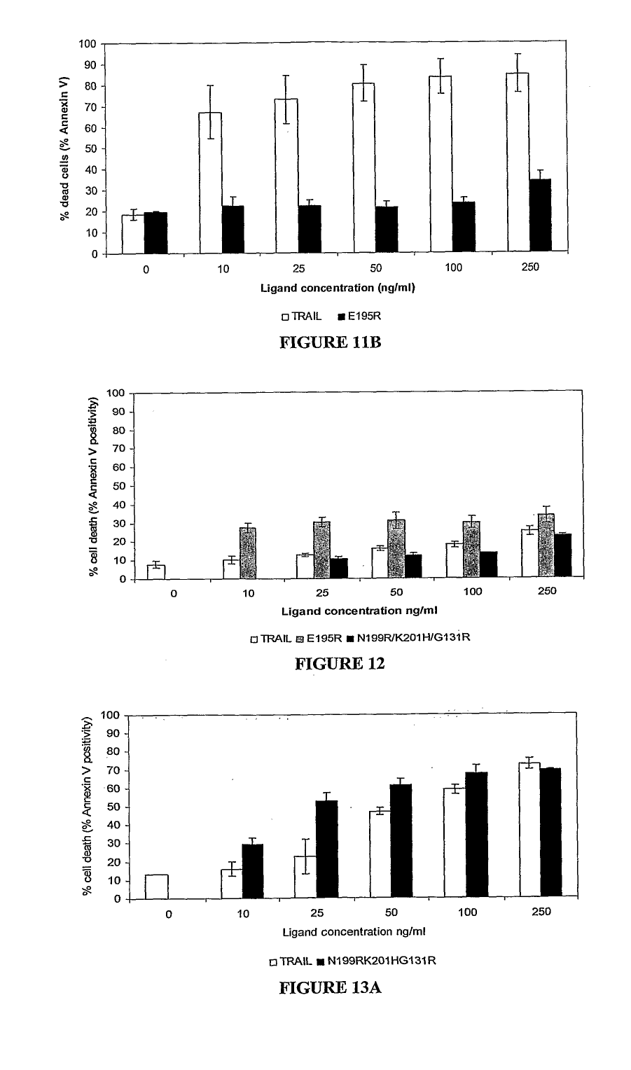

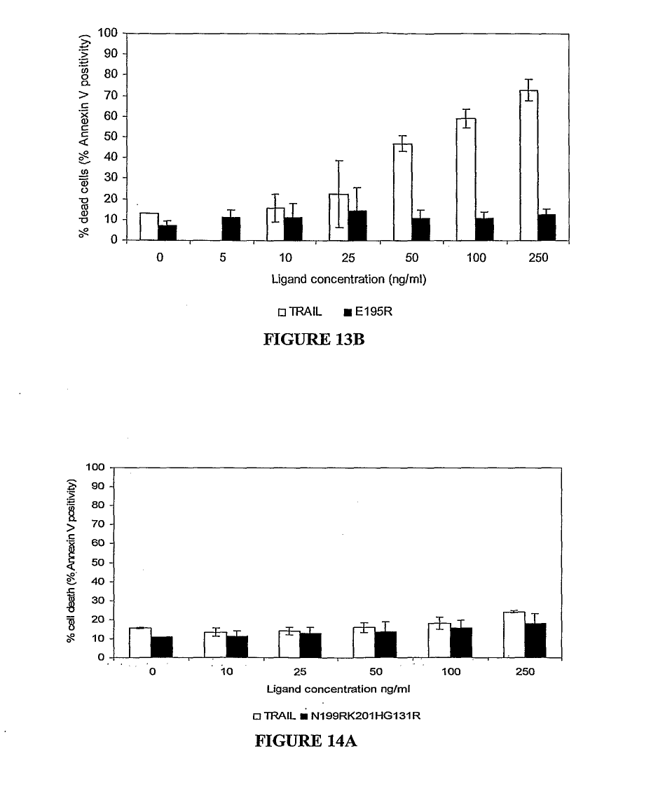

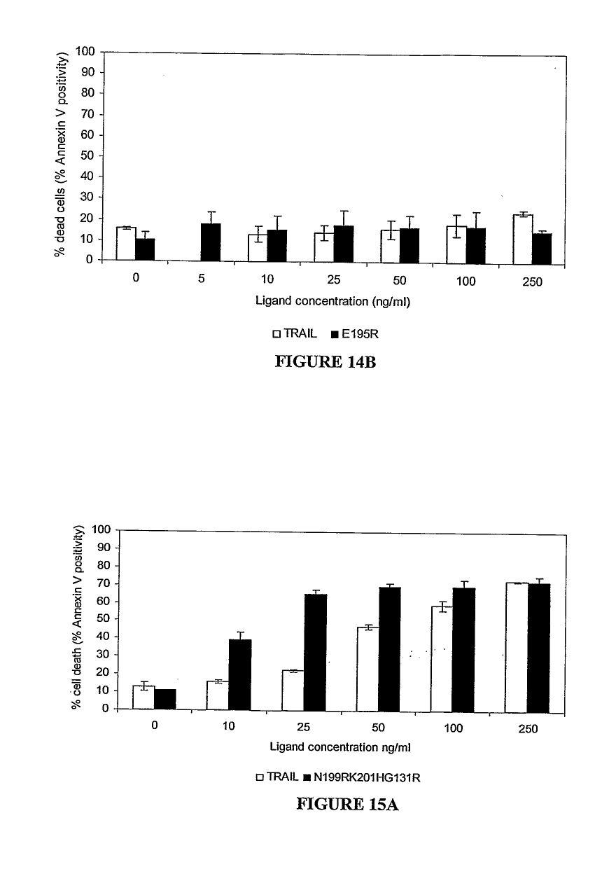

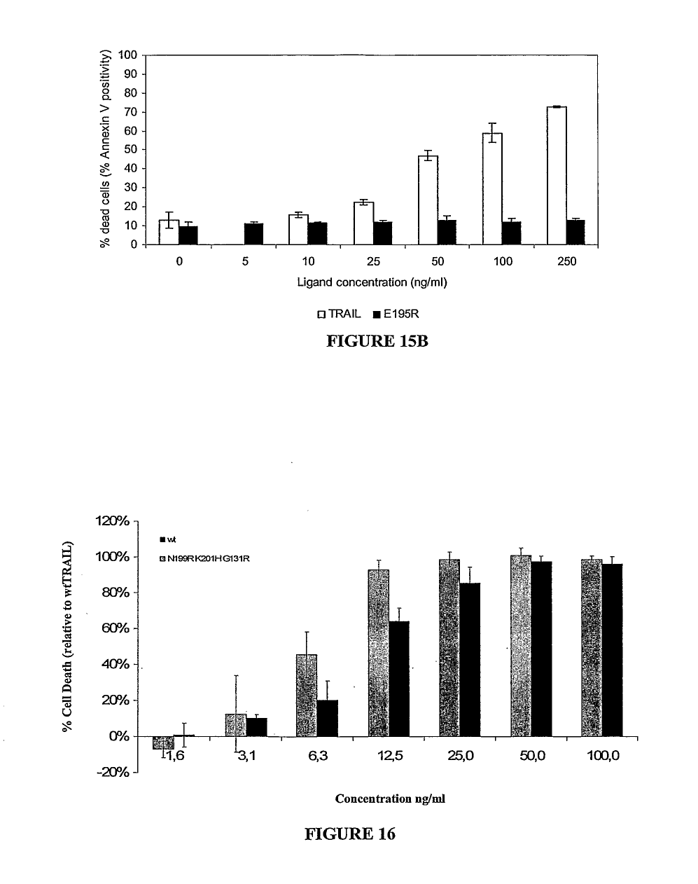

ML-1, EM-2 and A2780 cells were treated with a range of concentration of WT rhTRAIL, the candidate DR4-selective TRAIL variant N199R/K201H/G131R as well as D269H/E195R, a DR5-selective TRAIL variant (see WO05/056596). D269H/E195R had very little apoptosis-inducing activity in ML-1 and EM-2 cells, confirming the DR4-dependent nature of these cell lines (FIG. 10, 11). In contrast, D269H/E195R showed higher pro-apoptotic activity than WT rhTRAIL in A2780 cells, again confirming the exclusive, DR5-sensitivity of A2780 cells (FIG. 12). N199R/K201H/G131R on the contrary displayed higher pro-apoptotic activity than WT rhTRAIL in the DR4-responsive cell lines ML-1 and EM-2 (FIG. 10, 11) and only marginal activity in the A2780, DR5-responsive cells (FIG. 12). The colon cancer cell line Colo205 is known to express all four receptors on its cell surface and to mediate TRAIL induced apoptosis via DR4 and DR5²⁶. Accordingly, treatment with the DR4-selective variant resulted in an increased apoptotic efficacy in these cells (FIG. 16). These results indicate that N199R/K201H/G131R is an agonistic ligand that selectively binds to DR4.

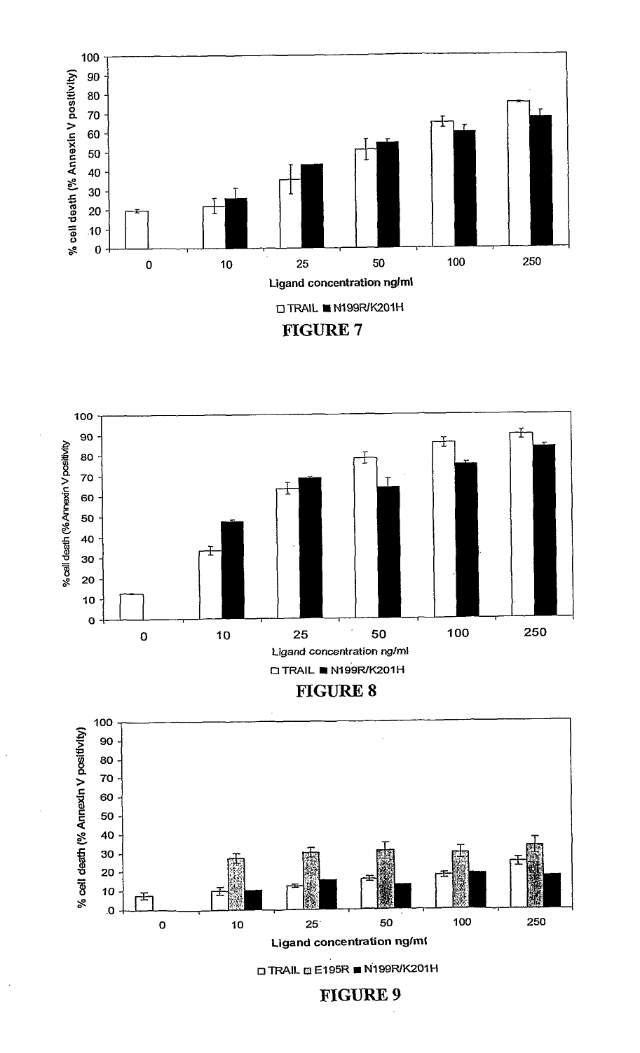

Similar testing was carried out with the double mutant N199K/K201H, also a predicted DR4-selective variant. N199K/K201H induced apoptosis to the same degree as WT rhTRAIL in ML-1 (FIG. 7) and EM-2 (FIG. 8) cells. Treatment with N199K/K201H induced significantly less apoptosis in A2780 cells in comparison to WT rhTRAIL, suggesting a DR4-selective agonistic activity (FIG. 9).

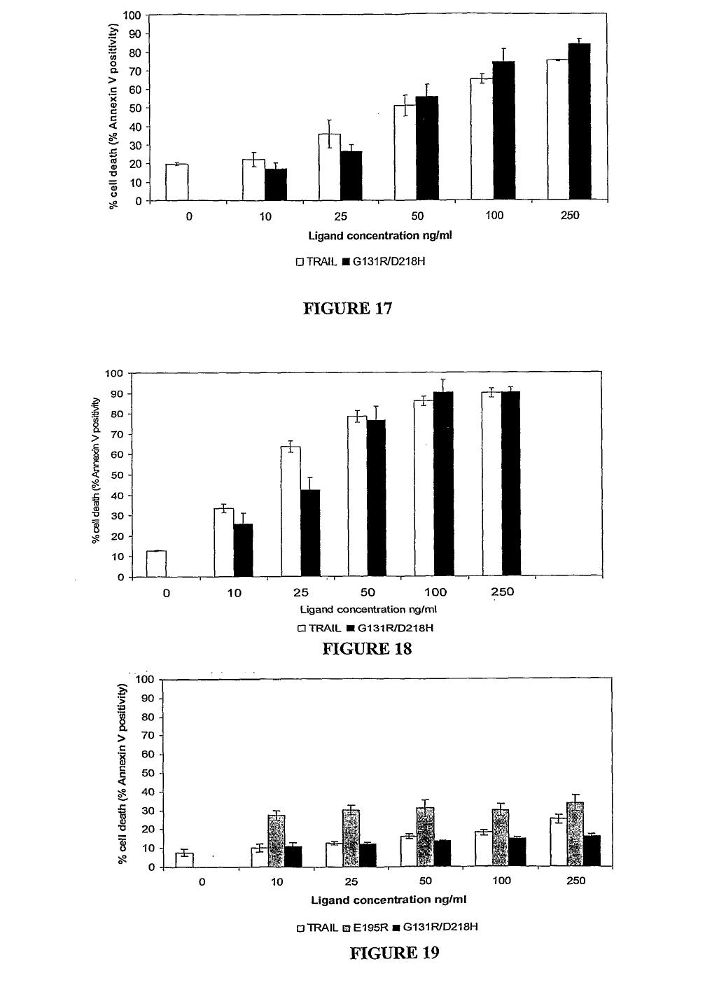

Testing of agonistic activity and receptor selectivity was then carried out with the double mutant G131R/D218H, also a predicted DR4-selective variant. G131R/D218H induced apoptosis to the similar degree as WT rhTRAIL in ML-1 cells (FIG. 17) and slightly reduced cytotoxicity in EM-2 cells (FIG. 18). On the contrary, treatment with G131R/D218H induced significantly less apoptosis in A2780 cells in comparison to WT rhTRAIL, suggesting a DR4-selective agonistic activity (FIG. 19).

### Example 7

### Biological Activity of G131R and G131R/N199R/K201H

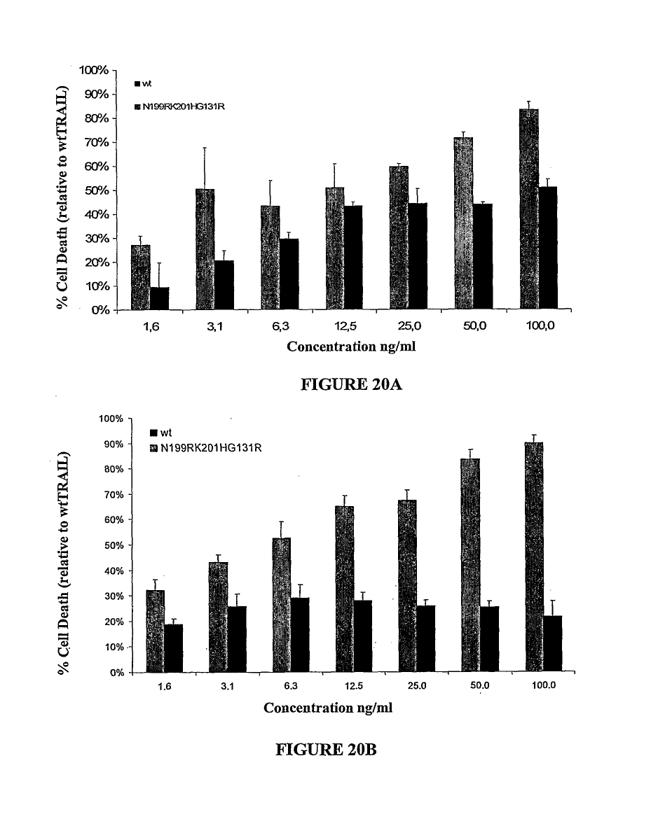

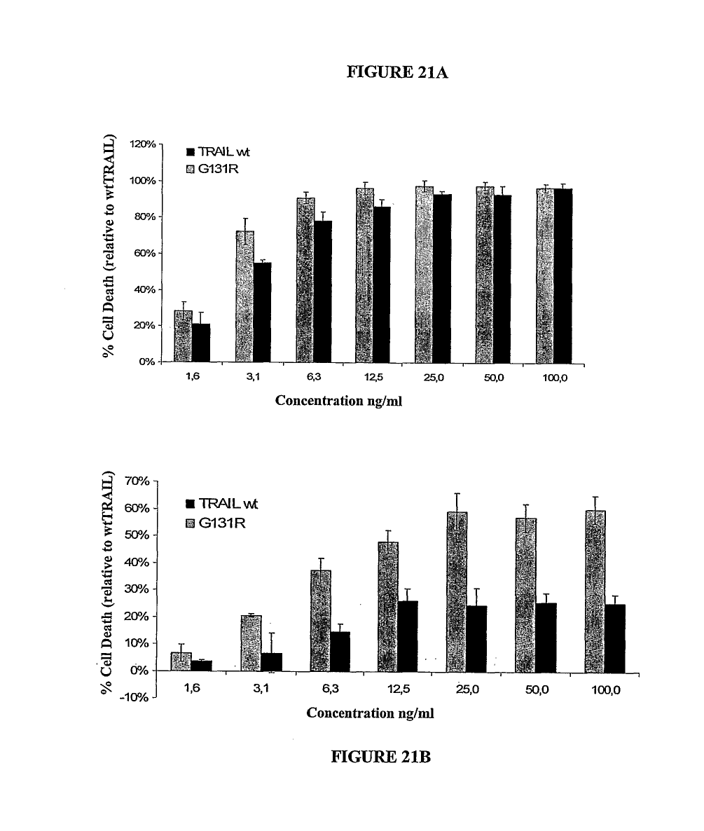

An additional experiment using the TRAIL variants G131R and G131R/N199R/K201H in wild-type BJAB cells responsive to both DR4- and DR5-mediated cell death (BJAB^{wt}) and BJAB cells deficient in DR5 (BJAB^{DR5DEF}) was performed. Both mutants were able to induce cell death in BJAB^{wt} cells (FIG. 20A, FIG. 21B) and in BJAB^{DR5DEF} cells (FIG. 20B, FIG. 21B). This indicates that these mutants show a high degree of DR4 specificity and that these mutants are strong agonists of the DR4 receptor.

### Example 8

### Biological Activity of Variant G131R/N199R/K201H

TRAIL variant G131R/N199R/K201H was tested for its biological activity in HL-60 cells. HL60 (acute myeloid leukaemia; DSMZ, ACC 3) were maintained in RPMI medium supplemented with 10% foetal bovine serum (FBS), 2 mM glutamine, 50 U penicillin and 50 mg/ml streptomycin. Cells were seeded at 300,000 cells/ml 24 h prior to treatment. Cells were cultured at 37° C. with 5% CO₂ in a humidified incubator. All reagents were from Sigma-Aldrich unless otherwise stated. To compare the ability of N199R/K201H/G131R and WT rhTRAIL to activate DR4, activation procaspase-8 and induction of apoptosis was measured after treating the cells with a 100 ng/ml of N199R/K201H/G131R WT rhTRAIL for the times indicated. At the indicated times, cells were harvested for Western blot analysis or washed, resuspended in complete medium in the absence of the ligands and analysed after a total incubation time of 12 h.

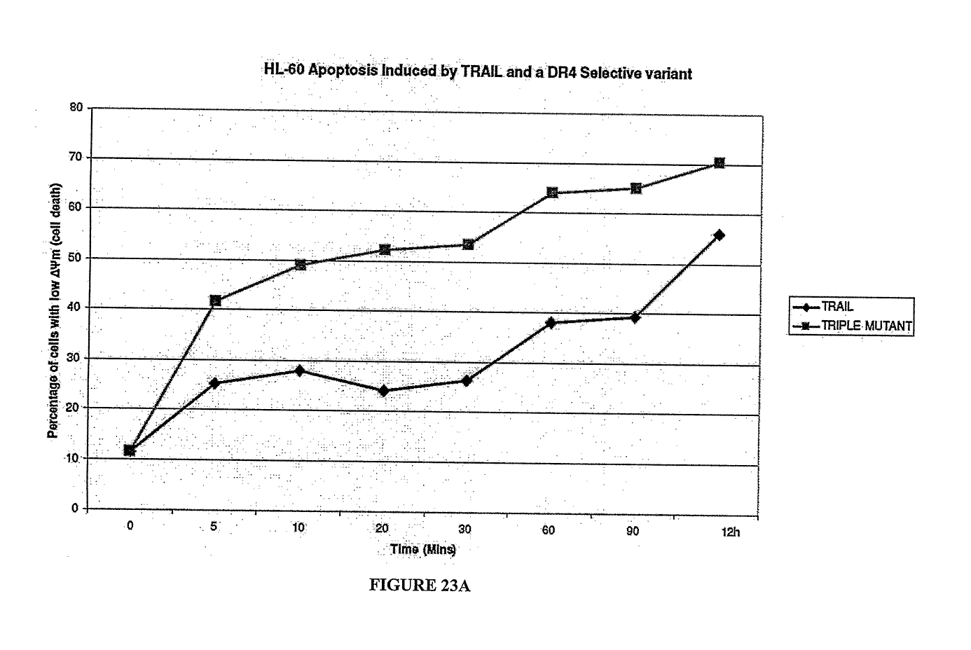

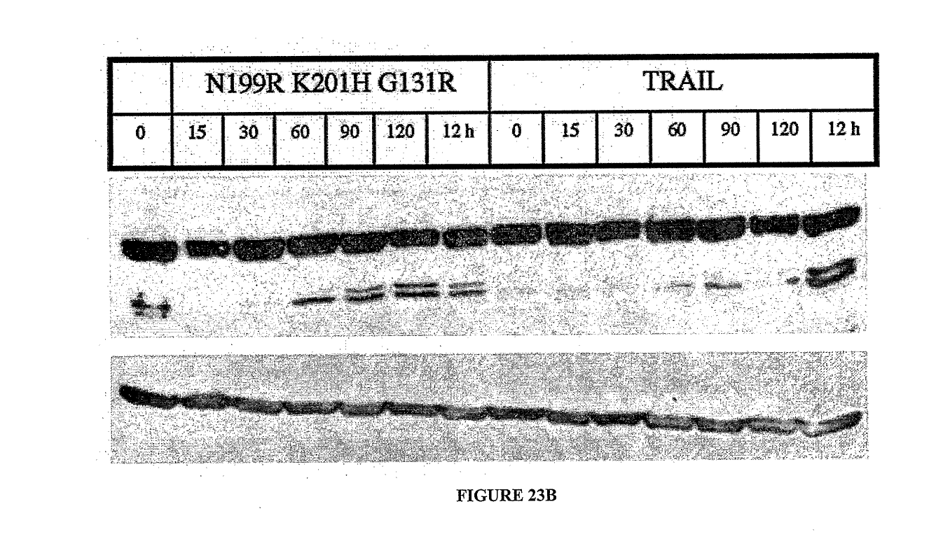

As can be seen in FIG. 23, variant N199R/K201H/G131R is a much stronger activator of DR4 than WT rhTRAIL shown both by processing of pro-caspase-8 (detected by Western blottin) and the level of cell death induced measured by determination of the number of cells with low mitochondrial membrane potential (FIGS. 23a and 23b).

### Example 9

Biological Activity of Variant N199R/K201H/G131R in Combination with BMS

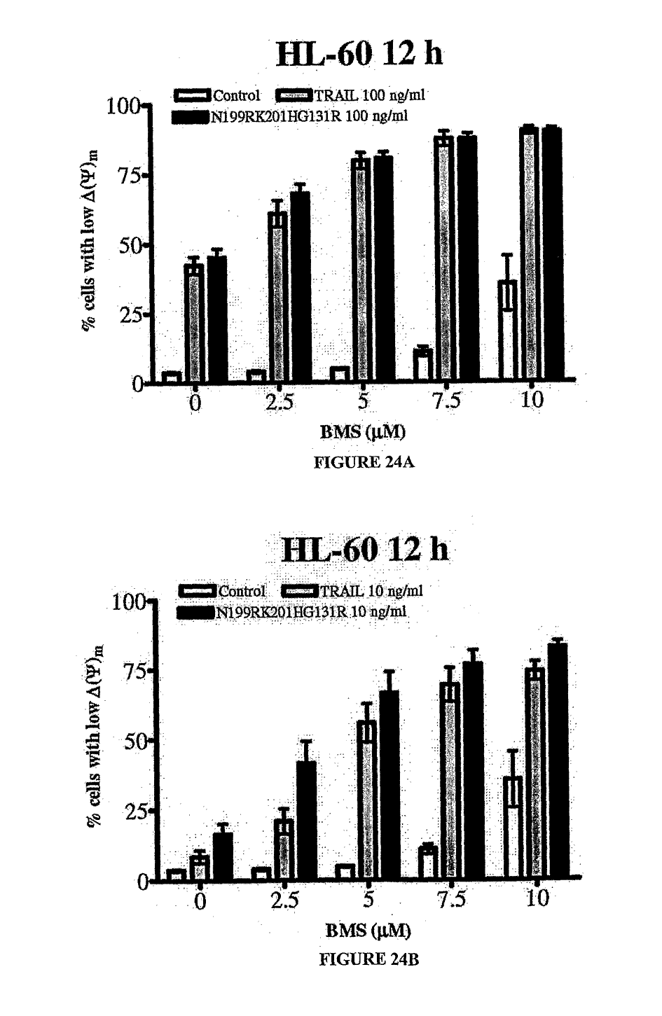

The triple mutant N199R/K201H/G131R is very efficient on myeloid cells and activates DR4 more efficiently than WT rhTRAIL (FIG. 23). Since both DR4 and DR5 can activate not only pr-caspase-8, but the anti-apoptotic NF-kB, the effect of NF-kB inhibition on the biological activity of both WT rhTRAIL and the variant N199R/K201H/G131R was tested. HI-60 cells were treated with WTrhTRAIL or N199R/K201H/G131R in the absence or presence of the Inhibitor kB kinase (IKK) inhibitor BMS-345541 for 12 h (FIG. 24). The increased efficacy of the triple combination with respect to WT rhTRAIL in presence of BMS is evident especially at lower concentrations of the ligands (10 ng/ml; FIG. 24b), which likely is an advantage for in vivo applications as anticancer treatments in view of the potentially toxic side effects of high doses of drugs.

### Example 10

### Binding Activity of R149I, S159R, K204Y, and S215D as Measured by SPR and Competitive ELISA

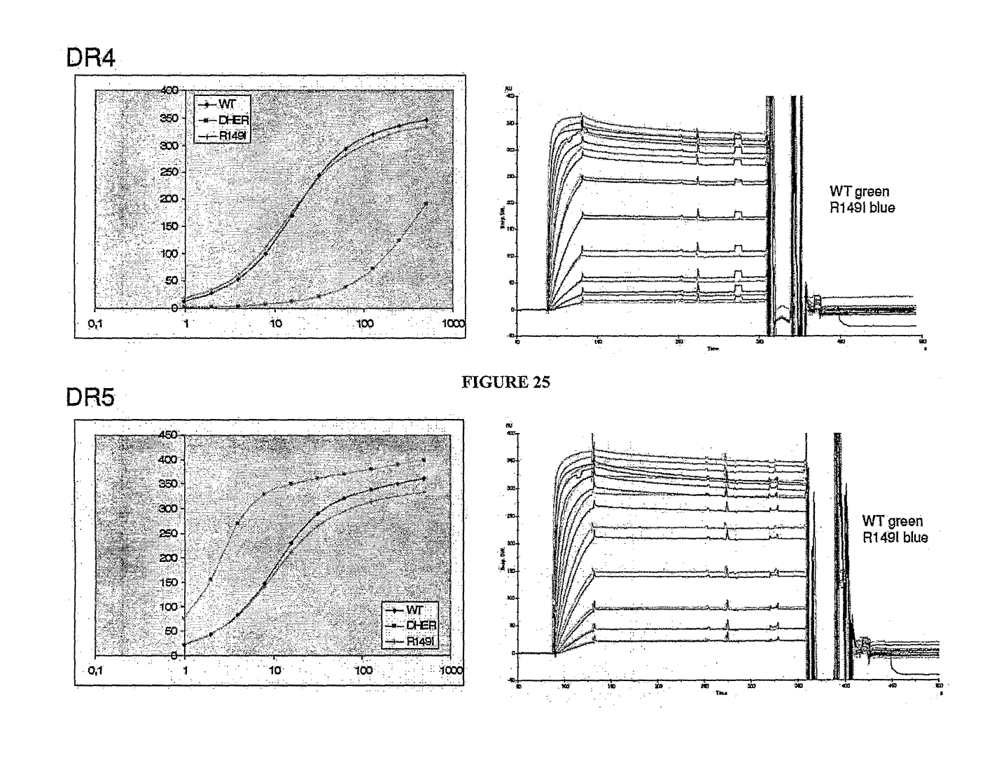

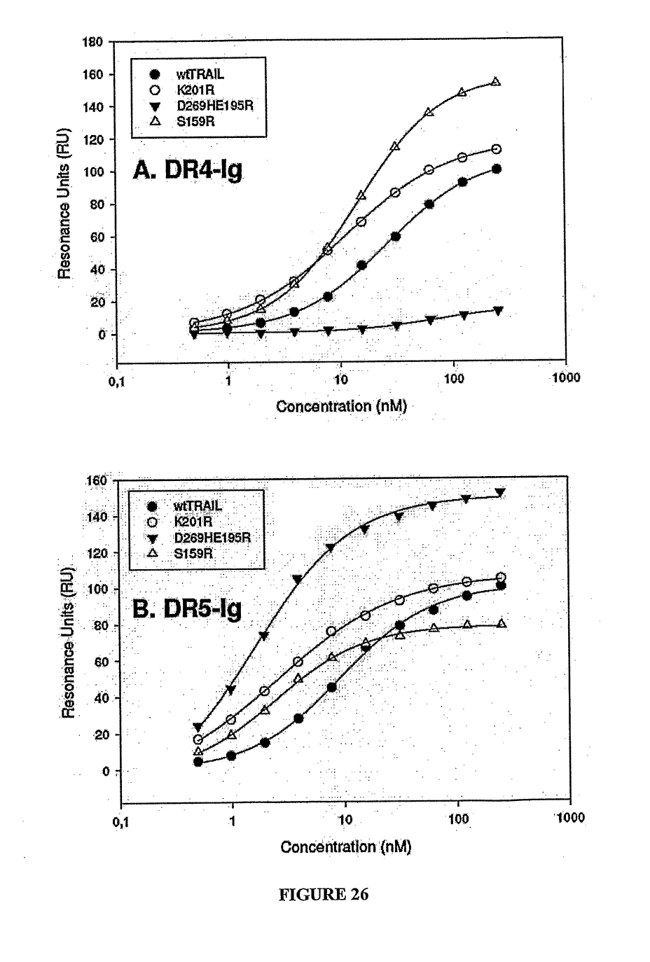

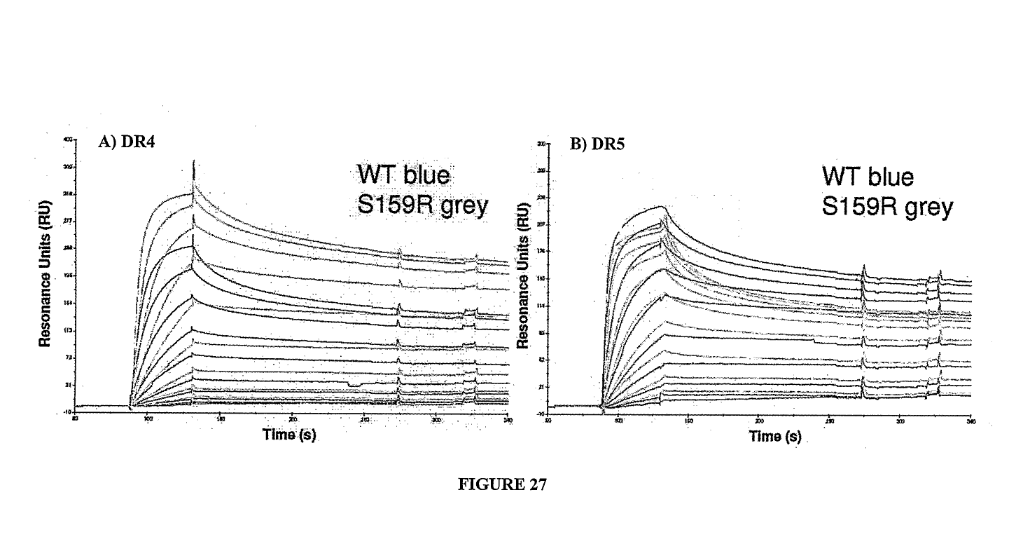

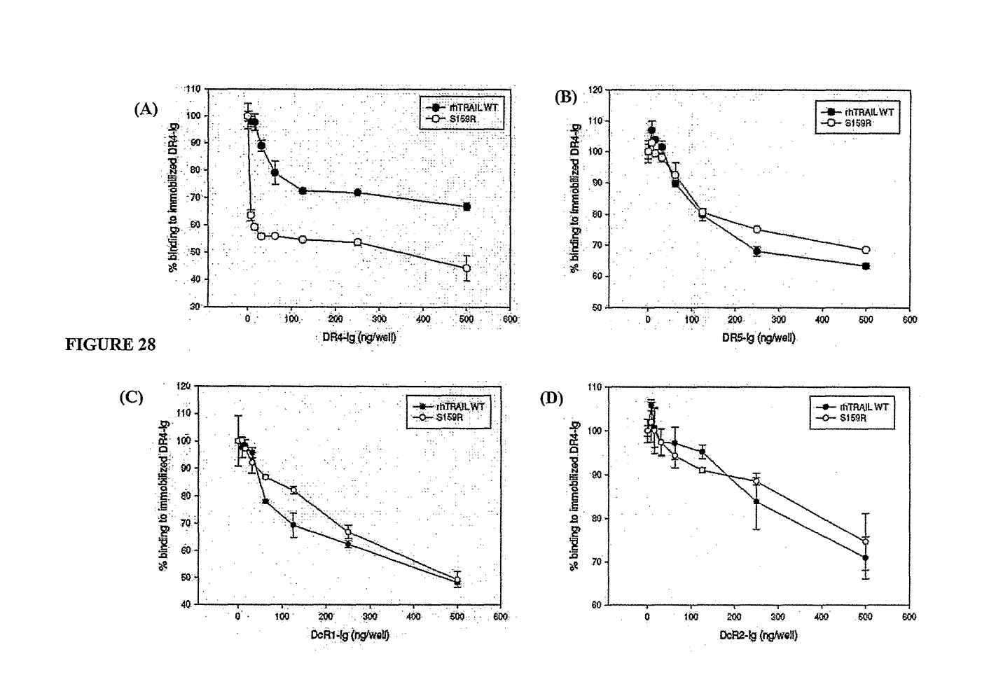

The SPR sensorgrams in FIG. 25 shows that the DR5 complex of variant R149I is destabilised whereas that of DR4 seems mostly unaltered, even when variant R149I does not significantly affect the pre-steady state affinities to both death receptors. Pre-steady state SPR measurements demonstrate that variant S159R is able to bind with higher apparent affinity to DR4-Ig (FIG. 26A), whereas the binding to DR5-Ig is reduced (FIG. 26B), thus clearly demonstrating DR4 specificity. A DR4-specificity is also demonstrated by variant K201R similarly, even when less pronounced (FIG. 26). The sensorgrams of variant S159R and WT rhTRAIL in FIG. 27 show that not only the apparent affinity but also the dynamics of the interaction with the receptor molecules is affected in favour of DR4. In addition, a competition ELISA experiment, as shown in FIG. 28, demonstrates that DR4-Ig and not DR5-Ig is able to compete to binding of the variant to immobilised DR4-Ig, again clearly demonstrating the DR4 specificity of variant S159R.

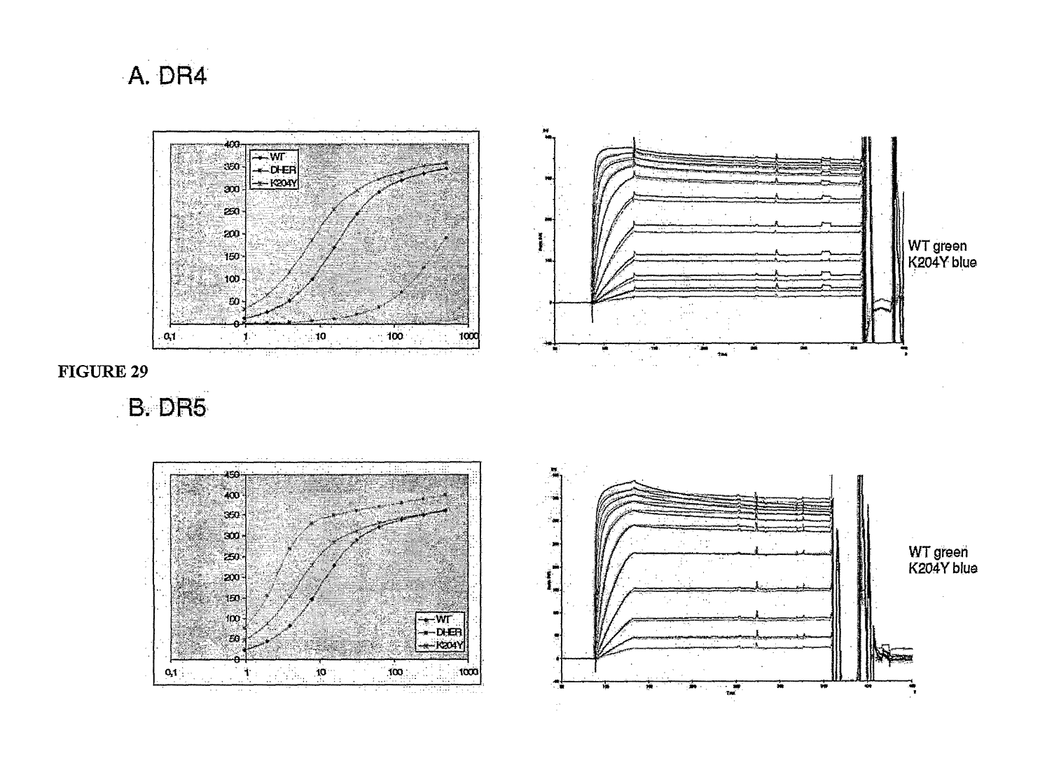

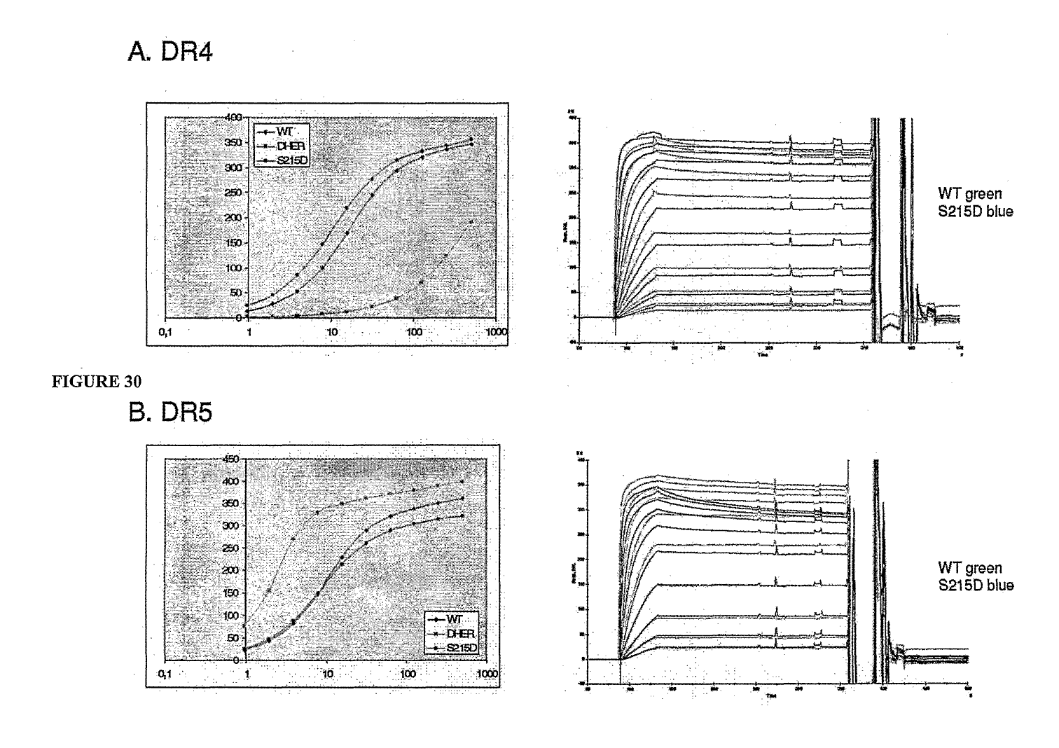

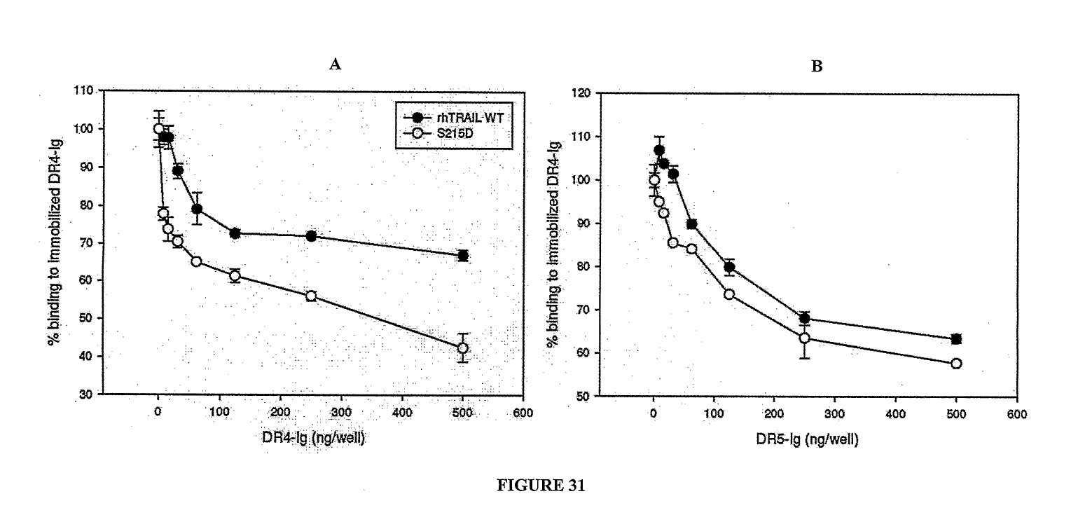

Mutations K204Y and S215D also affect the binding to receptors in favour of DR4. Whereas the binding to DR4-Ig as measured by pre-steady state SPR seems less dramatically affected, the complex with DR4-Ig seems to be stabilised and that of DR5-Ig destabilised (FIGS. 29 and 30). Variant S215D was further analysed by competition ELISA, where only DR4-Ig and not DR5-Ig is able to efficiently compete for the binding of S215D to immobilised DR4-Ig (FIG. 31), pointing at the DR4-specificity induced by this mutation.

### Example 11

### Biological Activity of S159R

The biological activity of TRAIL variant S159R has been tested on different cell lines. The human pancreatic carcinoma cell lines BxPc and Colo357 were maintained in RPMI medium supplemented with 10% foetal bovine serum (FBS), 2 mM glutamine, 50 U penicillin and 50 mg/ml streptomycin. HEPG2 (human hepatocellular carcinoma) cell line was maintained in DMEM medium supplemented with 10% FBS, 50 U penicillin and 50 mg/ml streptomycin. HCT116 WT and p53^{-/-} (human colon carcinoma) cell lines were maintained in McCoy's 5A medium with HEPES supplemented with 10% FBS, 50 U penicillin and 50 mg/ml streptomycin. All cells were cultured at 37° C. with 5% CO₂ in a humidified incubator. BxPc, Colo357 and HEPG2 were seeded at 150,000 cells/ml, HCT116 WT and p53^{-/-} were seeded at 200,000 cells/ml. Cells were seeded in three 96 well plates in a total volume of 100 μl/well. The day after seeding, cells were treated with a dosage (0-500 ng/ml) of WT rhTRAIL, D269H/E195R or S159R in triplicate. After 24 h incubation, one 96 well/plate (24 h time point) was used for MTT assay, the other two plates were treated again with the same concentrations of WT rhTRAIL, D269H/E195R and S159R and incubated for a further 24 h. After incubation, one 96 well/plate (48 h time point) was used for MTT assay and the other one (72 h time point) was treated as described above, incubated for further 24 h and analysed by MTT assay. After treatment, 10 μl of the MTT solution (5 mg/ml in Hanks' balanced salt solution) was added to the wells and the plate was incubated for 3 h at 37° C. with 5% CO₂ in a humidified incubator. After incubation, 100 μl of stop solution (20% SDS in 40% dimethyl formamide) was added to the wells and the plate was placed on an orbital shaker overnight to dissolve the formazan precipitate. After incubation, the absorbance was measured at 550 nm in a PerkinElmer Victor³ multiplate reader. Cell viability was expressed as average percentage of the control from three replicate wells.

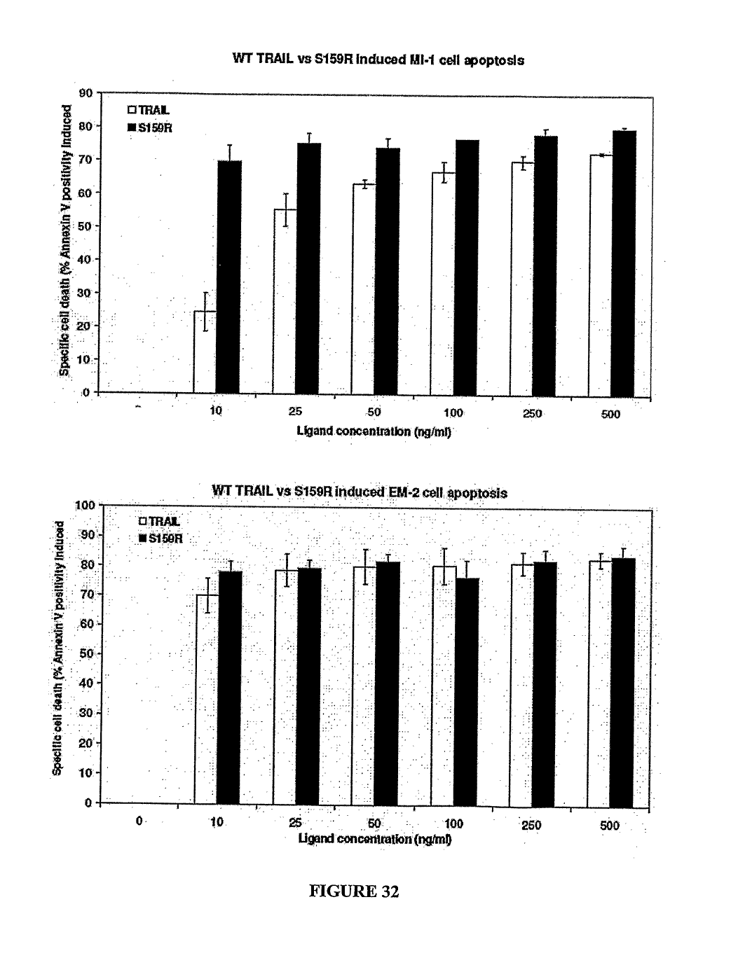

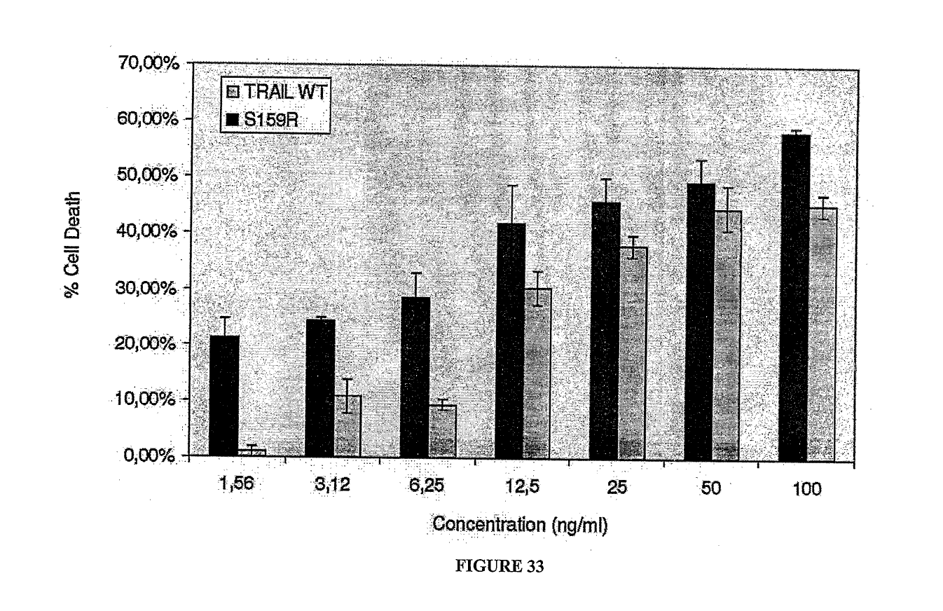

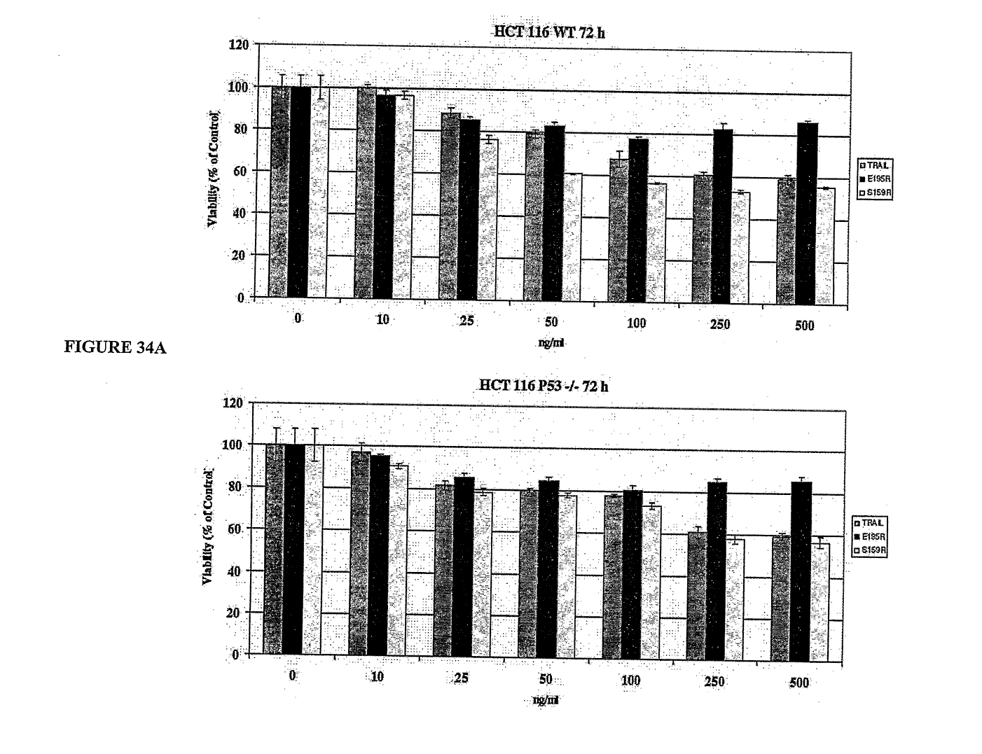

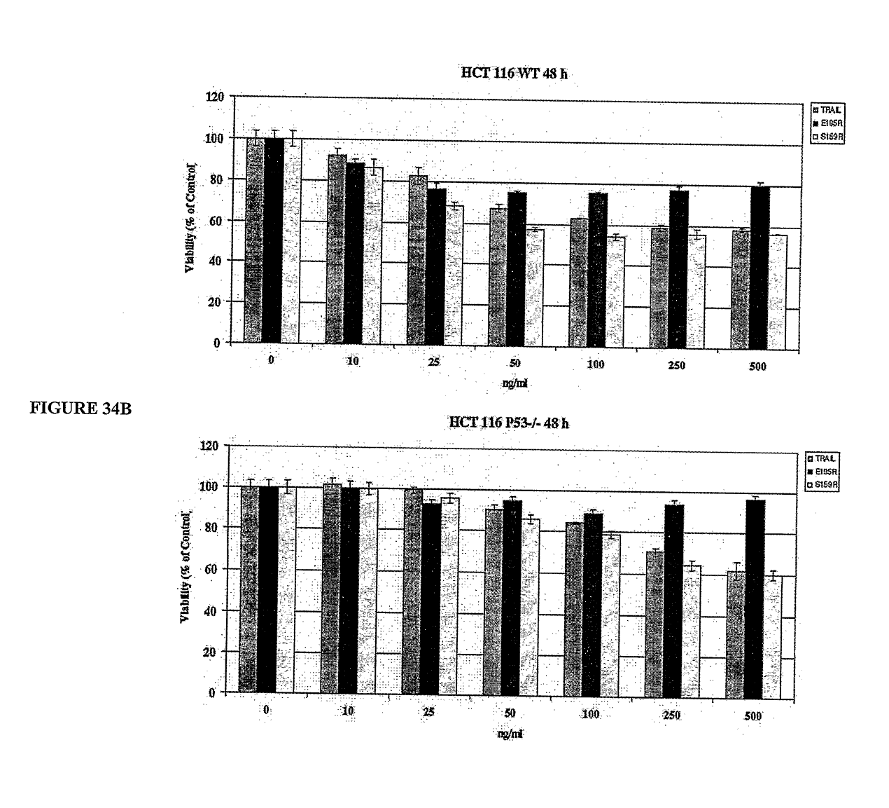

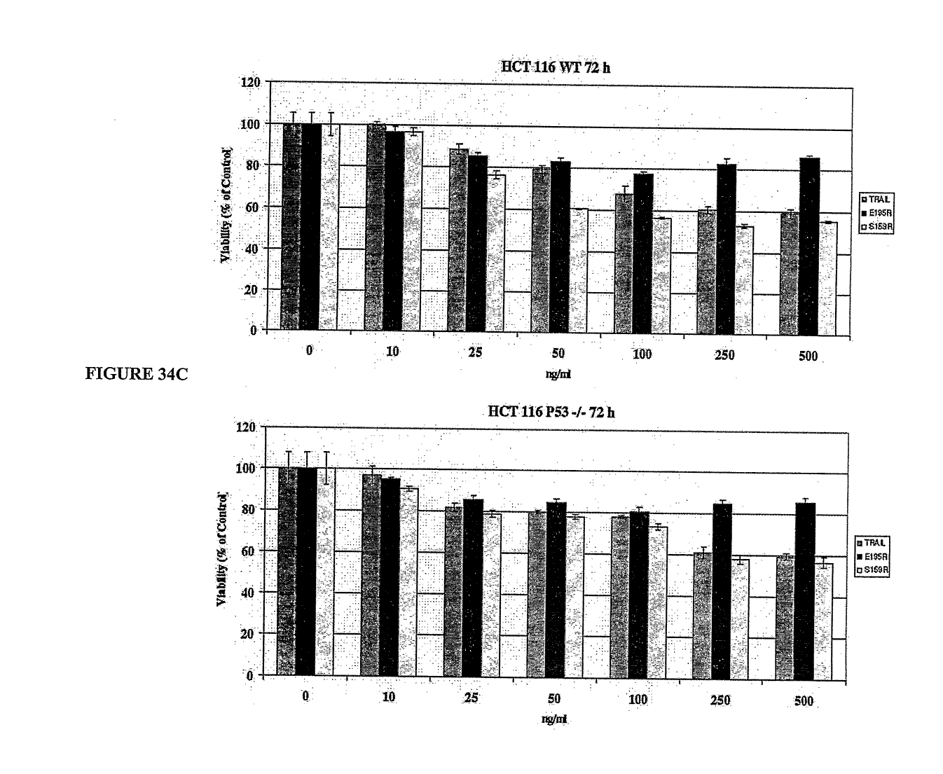

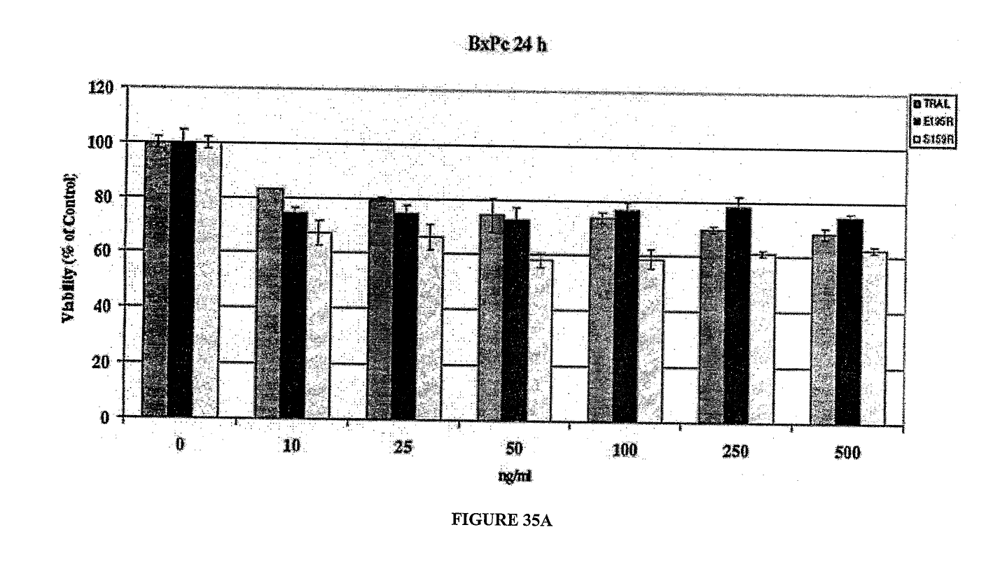

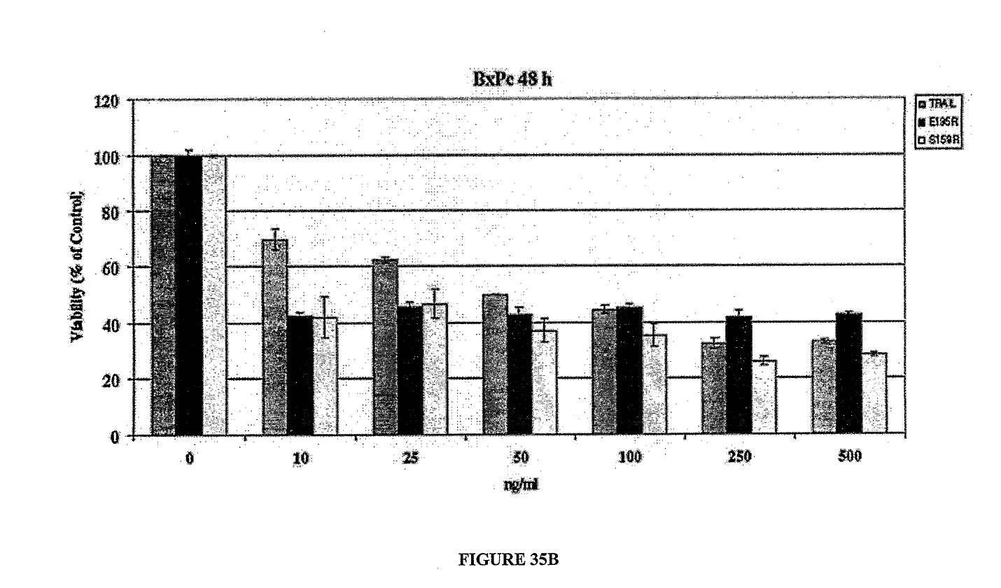

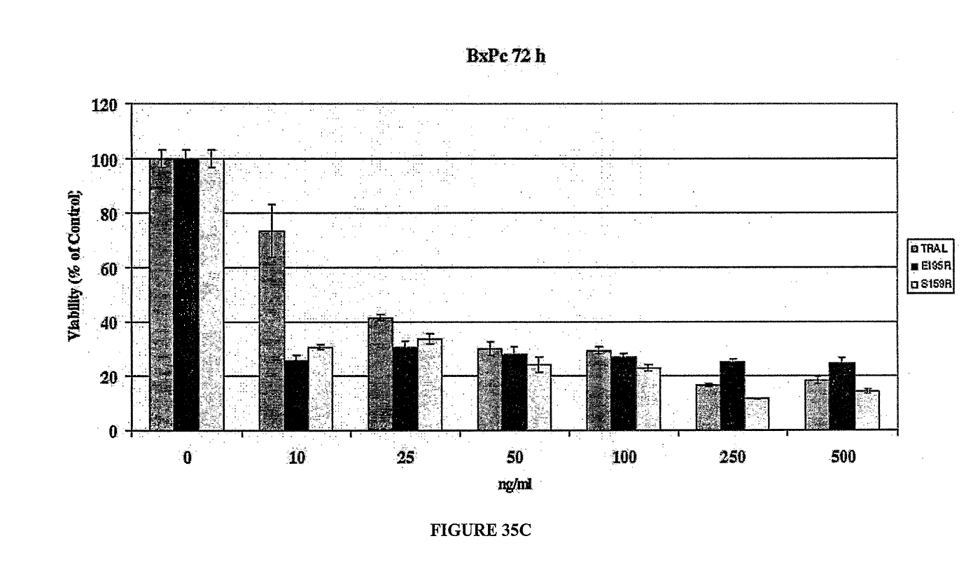

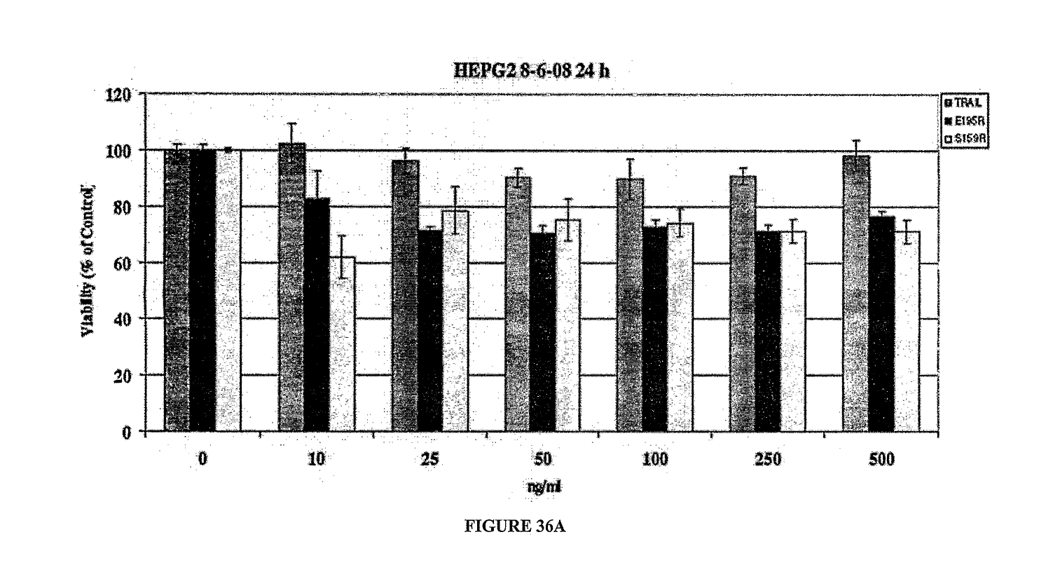

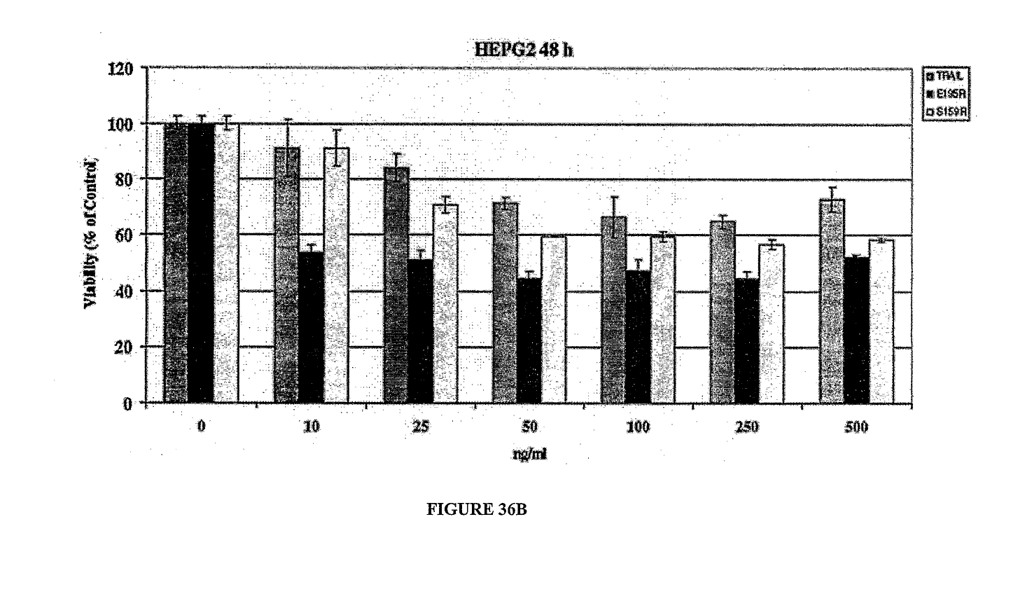

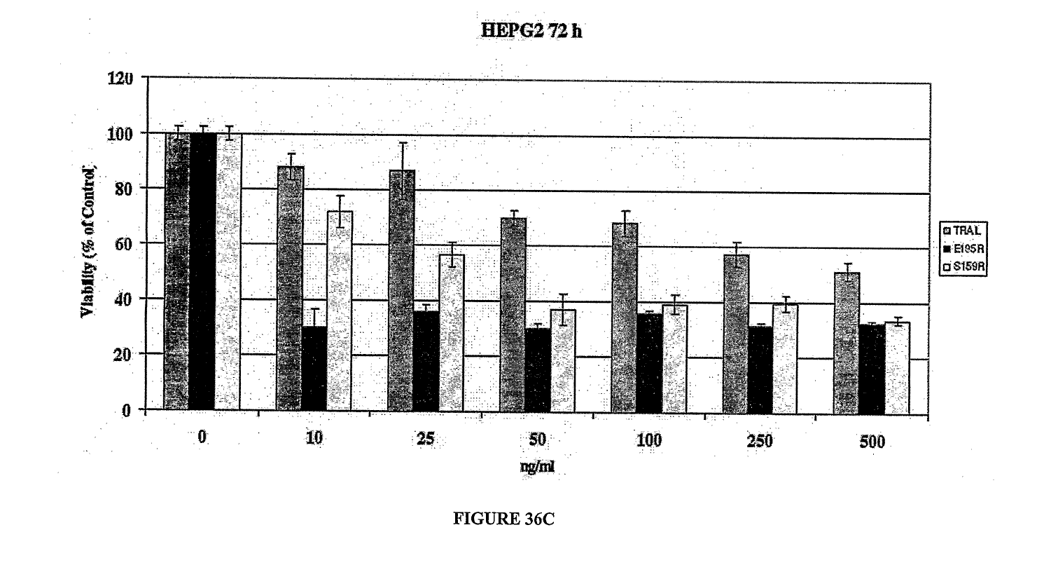

Variant S159R has an increased apoptotic activity on the DR4-sensitive cell lines ML-1 (FIG. 32, upper panel), EM-2 (FIG. 32, lower panel), both myeloid cell lines, demonstrating its DR4 selectivity. S159R also showed strong biological activity in the human colon carcinoma cell line, SW948 (FIG. 33). In all three cell lines the variant causes significant apoptosis already at very low concentrations, demonstrating the increased efficacy of this variant in comparison to WT rhTRAIL. The high efficacy of S159R in human colon carcinoma HCT116 (FIG. 34) underlines the high receptor-agonistic activity of this variant. The high efficacy of S159R was also demonstrated in the pancreatic carcinoma cell line BxPc that expresses both death receptors: DR4-selective variant S159R is equally active as DR5-specific variant D269H/E195R (FIG. 35). As expected, in cell line HepG2, where DR5 is the primary transducer of the TRAIL death signal, variant S159R is less active (FIG. 36).

In conclusion, variant S159R is a very efficacious, DR4-selective variant.

### Example 12

### Biological Activity of R149I and S215D

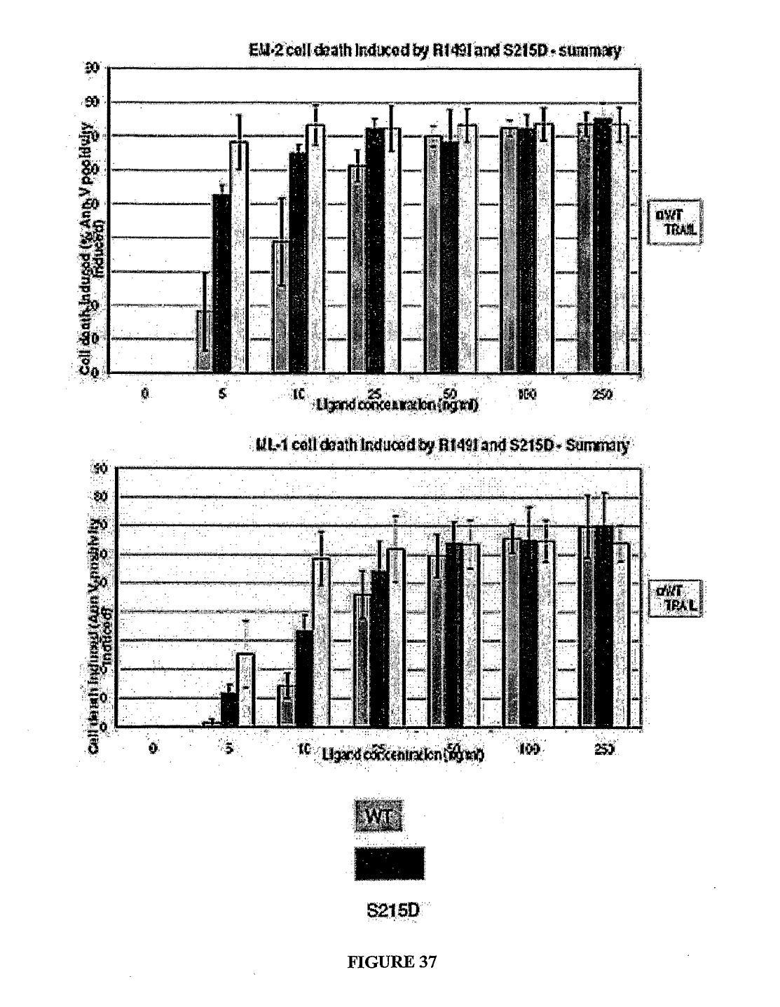

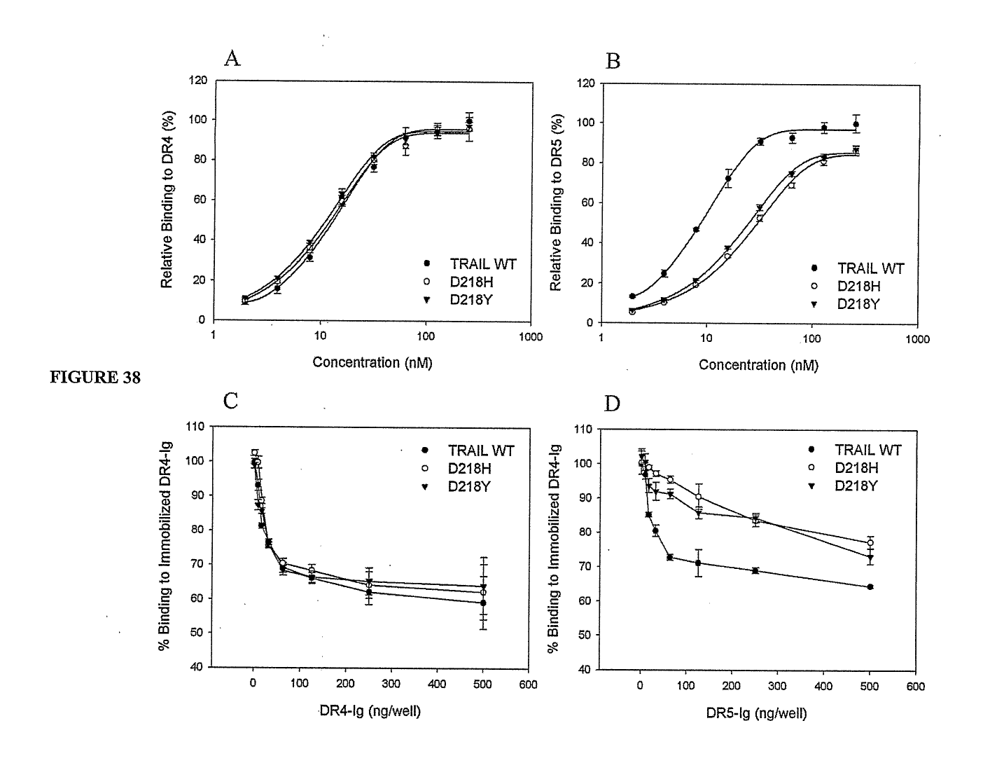

FIG. 37 demonstrates the biological activity of variants R149I and S215D in comparison to WT rhTRAIL on DR4-responsive myeloid cell lines EM-2 and ML-1. Clearly, both variants are more efficacious and induce apoptosis at lower concentrations than WT, the difference being approximately 5-fold in EM-2 and 2-fold in ML-1 cells indicating that these variants are strong agonists of DR4.

### Example 13

### Materials and Methods—Binding Experiments

Binding experiments were performed using a Surface Plasmon Resonance-based biosensor Biacore 3000 (Biacore AB). Research grade CM5 sensor chips, N-hydroxysuccimide (NHS), N-ethyl-N′-(3-diethylaminopropyl)carbodiimide (EDC), ethanolamine-HCl and standard buffers e.g. HBS-N and HBS-EP were purchased from the manufacturer. All the buffers were filtered and degassed. Immobilization of DR4-Ig and DR5-Ig receptors on the sensor surface of a Biacore CM5 sensor chip was performed following a standard amine coupling procedure according to the manufacturer's instructions. Receptors were coated at a level of ˜800 response units. Activated, coupled surfaces were then quenched of reactive sites with 1M ethanolamine (pH 8). Reference surfaces consisted of activated CM dextran, subsequently blocked with ethanolamine. A 50 μl aliquot of WT rhRAIL and variants were injected in three-fold at concentrations ranging from 250 nM to 0.5 nM at 70 μl/ml and at 37° C. using HBS-N supplemented with 0.005% surfactant P20 (GE Healthcare) as running and sample buffer. Binding of ligands to the receptors was monitored in real-time. Between injections the receptor/sensor surface was regenerated using 1:1 [10 mM glycine 1.5 M NaCl pH 2]/ethylene glycol and a contact time of 35 s.

### REFERENCES CITED

1 Boppana, S. B (1996), Indian. J. Pediatr. 63(4):447-52
2 Nishihira, J. (1998), Int. J. Mol. Med. 2(1):17-28
3 Kim, P. K. et al (2000), Surg. Clin. North. Am. 80(3):885-894
4 Clark, R. A. (1991), J. Cell Biochem. 46(1):1-2
5 Flad, H. D. et al (1999), Pathobiology. 67(5-6):291-293
6 Rodriguez, F. H. et al (2000), Curr. Pharm. Des. 6(6):665-680
7 Schmutzler, C. et al (2000), 143(1):15-24
8 Lazar-Molnar, E. et al (2000), Cytokine. 12(6), 547-554
9 Gado, K. (2000), 24(4):195-209
10 van Deventer, S. J. et al (2000), Intensive Care Med. 26 (Suppl 1):S98:S102
11 Shanafelt, A. B. et al (1998), 95(16):9454-9458
12 Ashkenazi et al (1999), J. Clin. Invest 104, 155-162
13 Ruiz de Almodóvar et al. (2003), November 17, Manuscript M311243200
14 Wiley et al (1995), Immunity. 3, 673-682
15 Pitti et al (1996), J Biol. Chem. 271, 12687-12690
16 Van der Sloot et al (2006), Proc Natl Acad Sci USA 103, 8634-8639
17 Terpe K (2003), Appl Microbiol Biotechnol, 60:523-33
18 Gentz et al. (1989), Proc Natl Acad Sci USA 86, 821-824
19 Wilson et al (1994), Cell 37, 767-778
20 Sambrook (1989), Molecular Cloning; A Laboratory Manual ISBN: 0879695773
21 Fernandez et al. (1998), Gene expression systems: Using nature for the art of expression ISBN: 0122538404
22 Summers et al. (1987), Texas Agricultural Experiment Station Bulletin No. 1555
23 Zenk (1991), Phytochemistry 30:3861-3863
24 Gennaro (2000), Remington: The Science and Practice of Pharmacy 20th ed, ISBN: 0683306472
25 Hymowitz et al. (2000), Biochemistry 39, 633-640
26 Van der Sloot (2006), PNAS 103(23): 8634-8639
27 MacFarlane M, Inoue S, Kohlhaas S L, Majid A, Harper N, Kennedy D B, Dyer M J, Cohen G M. Cell Death Differ. 2005; 12: 773-82

1. A variant TRAIL protein, which has superior selectivity for the death receptor 4 (TRAIL-R1) over the death receptor 5 (TRAIL-R2).

2. A variant TRAIL protein according to claim 1, which exhibits superior selectivity for the death receptor 4 (TRAIL-R1) over the decoy receptors DcR1 (TRAIL-R3) and DcR2 (TRAIL-R4).

3. A variant TRAIL protein according to claim 1, which has substantially greater affinity for the death receptor 4 (TRAIL-R1) over its affinity for the death receptor 5 (TRAIL-R2).

4. A variant TRAIL protein according to claim 2, which has substantially greater affinity for the death receptor 4 (TRAIL-R1) over its affinity for the decoy receptor DcR1 (TRAIL-R3) and/or DcR2 (TRAIL-R4).

5. A variant TRAIL protein according to claim 1, which has greater binding affinity for DR4 than does the wild type TRAIL molecule.

6. A variant TRAIL protein according to claim 1, which has lower binding affinity for DR5, DcR1 and/or DcR2 than does the wild type TRAIL molecule.

7. A variant TRAIL protein according to claim 1, which is mutated at one or more of the following positions selected from the group consisting of 131, 149, 159, 193, 199, 201, 204, 212, 215, 218 and 251.

8. A variant TRAIL protein according to claim 7, wherein the one or more mutation is selected from the group consisting of G131R, G131K, R149I, R149M, R149N, R149K S159R, Q193H, Q193K, N199H, N199R, K201H, K201R, K204E, K204D, K204L, K204Y, K212R, S215E, S215H, S215K, S215D, D218Y, D218H, K251D, K251E, and K251Q.

9. A variant TRAIL protein according to claim 7, in which both positions 199 and 201 are mutated.

10. A variant TRAIL protein according to claim 8, which contains the mutations N199R and K201H, or N199H and K201R.

11. A variant TRAIL protein according to claim 7, in which both positions 131 and 218 are mutated.

12. A variant TRAIL protein according to claim 11, which contains the mutations G131R and D218H.

13. A variant TRAIL protein according to claim 7, in which all of positions 131, 199 and 201 are mutated.

14. A variant TRAIL protein according to claim 13, which contains the mutations G131R, N199R and K201H.

15. A variant TRAIL protein according to claim 13, additionally comprising one or more mutations in positions 149, 159, 193, 204, 212, 215, 218 and 251.

16. A variant TRAIL protein according to claim 15, wherein the one or more mutation is in positions 149, 159 and 215.

17. A variant TRAIL protein according to claim 7 which is mutated at position 159 and at one or more positions selected from the group consisting of 149, 193, 204, 212, 215, 218 and 251.

18. A variant TRAIL protein according to claim 17, which is mutated at position 159 and at position 149 and/or 215.

19. A variant TRAIL protein according to claim 7, in which both positions 131 and 218 are mutated.

20. A variant TRAIL protein according to 7, in which one or more of the amino acids at positions 131, 199, 201, 215 and 218 are replaced by a basic amino acid.

21. A variant TRAIL protein according to claim 18, in which the basic amino acid is lysine, arginine or histidine.

22. A variant TRAIL protein according to claim 7, in which the amino acid at position 215 is replaced by an acidic amino acid.

23. A variant TRAIL protein according to claim 20, in which the acidic amino acid is glutamic acid or aspartic acid.

24. A variant TRAIL protein according to claim 1, which contains one or more of the mutations depicted in table 3.

25. A variant TRAIL protein according to claim 1, which is a fragment comprising a variant of amino acids 114-281 of the full length TRAIL sequence, optionally preceded by a methionine residue.

26. A TRAIL protein according to claim 25, which comprises a variant of the amino acid sequence presented in SEQ ID NO:3.

27. A variant TRAIL protein according to claim 1, which induces apoptosis in cancer cells.

28. A polynucleotide which encodes the variant TRAIL protein of claim 1.

29. A polynucleotide according to claim 28, which is: a variant of SEQ ID NO:2; a variant of nucleotides 88-933 of SEQ ID NO:2; or a variant of SEQ ID NO:4.

30. A host cell which expresses the polynucleotide of claim 28.

31. A method for inducing apoptosis in a mammalian cell comprising contacting said cell with the variant TRAIL protein of claim 1.

32. The method of claim 31 wherein the mammalian cell signals primarily through the DR4 specific pathway.

33. The method of claim 31, wherein the mammalian cell is a cancer cell.

34. The method of claim 33, wherein the cancer cell is an ovarian cancer, colon cancer or leukaemia cell.

35. The method of claim 34, wherein the mammalian cell is a human cell.

36. A pharmaceutical composition comprising a TRAIL variant according to claim 1.
